(19) United States
(12) Patent Application Publication
(10) Pub. No.: US 2025/0135087 A1
(43) Pub. Date: May 1, 2025

(54) METHOD FOR DETECTING EXTERNAL LIGHT WITHIN OPHTHALMIC SURGICAL CASSETTES

(71) Applicant: Alcon Inc., Fribourg (CH)

(72) Inventors: Jiansheng ZHOU, Irvine, CA (US); Brian William MCDONELL, Irvine, CA (US); John PULERA, Lake Forest, CA (US); Omid S. ROHANI, Irvine, CA (US)

(21) Appl. No.: 18/918,638

(22) Filed: Oct. 17, 2024

Related U.S. Application Data
(60) Provisional application No. 63/594,718, filed on Oct. 31, 2023.

(51) Int. Cl. A61M 1/00; A61F 9/007; G06K 7/14; H04N 23/71

(52) U.S. Cl. CPC A61M 1/72; A61F 9/007; G06K 7/1417; G06K 7/1443; H04N 23/71; A61B 2217/005; A61B 2217/007; A61M 2205/18; A61M 2205/3306; A61M 2205/6072

(57) ABSTRACT

Embodiments disclosed herein provide a surgical console, a surgical cassette, and a method for utilizing an image sensor to acquire grayscale image(s) and an embedded microcontroller to then process the image(s) to decode the barcode and/or determine the current level of fluid within the surgical cassette. The image sensor is utilized to detect external light that comes into the field of view of the image sensor. A software algorithm stored within a microcontroller in the surgical console determines whether the potential is high for external light interference. If there is a high potential for external light interference, an advisory or alarm is generated to warn the user to remove the external light source or to turn the console away from the external light source. Once the user has removed the external light and then cleared the advisory, normal console operations and functions can resume.

### CROSS-REFERENCE TO RELATED APPLICATIONS

This application claims the benefit of U.S. Provisional Application Ser.

No. 63/594,718 (filed on Oct. 31, 2023), the content of which is incorporated herein by reference in its entirety.

### INTRODUCTION

The present disclosure relates generally to ophthalmic surgical cassettes, and method of use thereof.

### BRIEF SUMMARY

The present disclosure relates generally to ophthalmic surgical consoles and cassettes, and methods of use thereof.

The following description and the related drawings set forth in detail certain illustrative features of one or more embodiments.

### BRIEF DESCRIPTION OF THE DRAWINGS

The appended figures depict certain aspects of one or more disclosed embodiments and are therefore not to be considered limiting of the scope of this disclosure.

FIG. 1A illustrates an example of an ophthalmic surgical system that may be used to perform ophthalmic procedures on an eye, according to certain embodiments.

FIG. 1B is an example of subsystems of a console of the ophthalmic surgical system of FIG. 1A, according to certain embodiments.

FIG. 2A is a back side isometric view of an example surgical cassette which may be operably coupled to a console of an ophthalmic surgical system, according to certain embodiments.

FIG. 2B is a back side elevation view of the surgical cassette of FIG. 2A, according to certain embodiments.

FIG. 3 is a magnified cross sectional schematic view of the interaction between a surgical cassette and a surgical console, which illustrates an image sensor disposed in the surgical console detecting a barcode disposed on the surgical cassette that is illuminated by a light source disposed within the surgical console, according to certain embodiments.

FIG. 4 illustrates a final image as obtained by the image sensor seen in FIG. 3 of the barcode disposed on the surgical cassette, according to certain embodiments.

FIG. 5A illustrates an image of the barcode on the surgical cassette as obtained by the image sensor and used to produce the final image seen in FIG. 4 when a lower left visible light source remains darkened, according to certain embodiments.

FIG. 5B illustrates another image of the barcode on the surgical cassette as obtained by the image sensor and used to produce the final image seen in FIG. 4 when an upper right visible light source remains darkened, according to certain embodiments.

FIG. 5C illustrates another image of the barcode on the surgical cassette as obtained by the image sensor and used to produce the final image seen in FIG. 4 when a lower right visible light source remains darkened, according to certain embodiments.

FIG. 5D illustrates another image of the barcode on the surgical cassette as obtained by the image sensor and used to produce the final image seen in FIG. 4 when an upper left visible light source remains darkened, according to certain embodiments.

FIG. 6 is another magnified cross sectional schematic view of the interaction between a surgical cassette and a surgical console, this time illustrating an image sensor disposed in the surgical console detecting a fluid level within the surgical cassette by illuminating a reservoir with infrared light, according to certain embodiments.

FIG. 7A is an image of a fluid level window captured by the image sensor corresponding to when the reservoir in FIG. 6 is empty, according to certain embodiments.

FIG. 7B is an image of a fluid level window captured by the image sensor corresponding to when the reservoir in FIG. 6 is ¼ full, according to certain embodiments.

FIG. 7C is an image of a fluid level window captured by the image sensor corresponding to when the reservoir in FIG. 6 is ½ full, according to certain embodiments.

FIG. 7D is an image of a fluid level window captured by the image sensor corresponding to when the reservoir in FIG. 6 is ¾ full, according to certain embodiments.

FIG. 8 is a flow diagram illustrating a method for operating the image sensor of FIG. 3, wherein a test or check for external light interference is performed before a barcode of the surgical cassette is decoded, according to certain embodiments.

FIG. 9 is a flow diagram illustrating a method for checking for external light interference within the method of FIG. 8, wherein an average pixel brightness value for an image is calculated, according to certain embodiments.

FIG. 10 is a flow diagram illustrating a method for checking for external light interference within the method of FIG. 8, wherein average pixel brightness values of each image within a limited plurality of images in consecutive frames are calculated, according to certain embodiments.

FIG. 11 is a flow diagram illustrating a method for checking for external light interference within the method of FIG. 8, wherein average pixel brightness values of each image within a large plurality of images in consecutive frames are calculated, according to certain embodiments.

FIG. 12A is a flow diagram illustrating a method for checking for external light interference within the method of FIG. 8, wherein a map for all pixels that have pixel brightness values that are equal to or greater than a predetermined threshold for an image is generated, according to certain embodiments.

FIG. 12B is an image of the barcode area comprising a fluid level window captured by the image sensor showing local regions of pixel brightness value that are equal to or greater than a predetermined threshold and with individual sizes that are larger than a predetermined limit, which can be mapped and processed for determining external light interference by the method of FIG. 12A, according to certain embodiments.

FIG. 12C is an image of the barcode area comprising a fluid level window captured by the image sensor showing local regions of pixel brightness value that are equal to or greater than a predetermined threshold and with individual sizes that are smaller than a predetermined limit which can be mapped and processed for determining external light interference by the method of FIG. 12A, according to certain embodiments.

FIG. 13 is a flow diagram illustrating a method for checking for external light interference within the method of FIG. 8, wherein a limited plurality of maps for all pixels that have pixel brightness values that are equal to or greater than a predetermined threshold for a corresponding limited plurality of images in consecutive frames are generated, according to certain embodiments.

FIG. 14 is a flow diagram illustrating a method for checking for external light interference within the method of FIG. 8, wherein a large plurality of maps for all pixels that have pixel brightness values that are equal to or greater than a predetermined threshold for a corresponding large plurality of images in consecutive frames are generated, according to certain embodiments.

FIG. 15A is a flow diagram illustrating a method for operating the image sensor of FIG. 3, wherein a check for external light interference is performed after a failure of decoding a barcode of the surgical cassette, according to certain embodiments.

FIG. 15B is an image of the barcode area comprising a fluid level window of the surgical cassette captured by the image sensor when external light is interfering with the barcode, according to certain embodiments.

FIG. 16 is a flow diagram illustrating a method for operating the image sensor of FIG. 3, wherein a check for external light interference is performed only after a predetermined number of failures of decoding a barcode of the surgical cassette using different decoding parameters, according to certain embodiments.

FIG. 17A is a flow diagram illustrating a method for operating the image sensor of FIG. 3, wherein a check for external light interference is performed after successfully decoding a barcode of the surgical cassette, according to certain embodiments.

FIG. 17B is an image of the barcode area comprising a fluid level window of the surgical cassette captured by the image sensor when external light is interfering with the fluid level window, according to certain embodiments.

FIG. 18 is a flow diagram illustrating a method for operating the image sensor of FIG. 3, wherein a check for external light interference is performed after a failure to calibrate the fluid level sensor against the surgical cassette, according to certain embodiments.

FIG. 19 is a flow diagram illustrating a method for operating the image sensor of FIG. 3, wherein a check for external light interference is periodically performed after successfully decoding the barcode and calibrating the fluid level sensor against the surgical cassette while the surgical console is in a stand-by mode, according to certain embodiments.

FIG. 20 is a flow diagram illustrating a method for operating the image sensor of FIG. 3, wherein a check for external light interference is periodically performed after successfully decoding the barcode and calibrating the fluid level sensor against the surgical cassette while the surgical console is in a surgery mode, according to certain embodiments.

FIG. 21A is a flow diagram illustrating a method for operating the image sensor of FIG. 3, wherein an average pixel brightness value within a defined checking region of each image within continuous series of live frames is calculated for checking external light interference while the fluid level of the surgical cassette is being actively monitored, and while the surgical console is in either a stand-by or surgery mode, according to certain embodiments.

FIG. 21B is an image of the barcode area comprising a fluid level window of the surgical cassette captured by the image sensor showing a defined checking region outside of the barcode area comprising a fluid level window for checking external light interference, according to certain embodiments.

FIG. 21C is an image of the barcode area comprising a fluid level window of FIG. 21B when strong external light is present within the defined checking region for checking external light interference, the external light being defined by an average pixel brightness value of the defined checking region is greater than a predetermined threshold, according to certain embodiments.

FIG. 22A is a flow diagram illustrating a method for operating the image sensor of FIG. 3, wherein continuous mapping of local regions of pixel brightness value that are equal to or greater than a predetermined threshold within a defined checking region of each image within a continuous series of live frames is performed for checking external light interference while the fluid level of the surgical cassette is being actively monitored and while the surgical console is in either a stand-by or surgery mode, according to certain embodiments.

FIG. 22B is an image of the barcode area comprising a fluid level window of FIG. 21B when external light is present within the defined checking region, the external light being defined as local regions of pixel brightness value that are greater than a predetermined threshold and wherein the area of these local regions are individually larger than a predetermined limit, according to certain embodiments.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

During ophthalmic surgery, a surgical cassette may be operably coupled with a fluidics module of a surgical console and used to facilitate the aspiration, suction, irrigation, or infusion functionalities typically performed during an ophthalmic surgery. The surgical cassette typically has one or more fluid chambers or reservoirs, for example an irrigation chamber, an infusion chamber, an aspiration chamber, and a Venturi chamber. A barcode is typically printed or marked on the surgical cassette which stores specific data/information related to the surgical cassette including pressure sensor calibration pressure sensor data or parameters, cassette identification, and manufacturing date. In general, during surgical console setup, after the surgical cassette has been successfully coupled to the fluidics module of the surgical console, a barcode reader of the surgical console decodes the data or information stored in the barcode. The surgical console then performs calibration(s) for one or more fluid level sensors against the surgical cassette. After console setup, working with one or more fluid level sensors of the surgical console in conjunction with the one or more valve assemblies, one or more peristaltic pumps, and/or a Venturi pump, the surgical console tracks and maintains proper fluid levels in the fluid chambers of the cassette in console stand-by mode and during surgical procedures.

However, in conventional surgical consoles the barcode reader and the fluid level sensors are optical based and operate under internal illumination sources within the surgical console. Therefore, any external light which penetrates the surgical cassette and seen by either the barcode reader and/or the fluid level sensors is effectively unwanted noise. Depending on its intensity, when external light gets inside the surgical cassette, it may not only interfere with the ability of the surgical console to decode the barcode on the surgical cassette currently coupled to the fluidics module, but it may also interfere with the ability of the surgical console to accurately read the current fluid levels within the fluid chambers of the surgical cassette resulting in losing proper control of fluid levels and consequently disabling other fluid level dependent console functions.

Therefore, there is a need for detecting external light within the surgical cassette and further determining if it may interfere with proper operations of barcode reader and/or fluid level sensors. Once an external light interference is determined, the external light source is removed or otherwise mitigated, thereby ensuring that the surgical console's proper functions of decoding the barcode and accurately reading fluid levels within the fluid chambers of the surgical cassette are completed.

Accordingly, certain embodiments disclosed herein provide an ophthalmic surgical console comprising a level sensor barcode reader (LSBR) in its fluidics module. The LSBR is an optical sensor utilizing a complementary metal-oxide semiconductor (CMOS) sensor camera to capture grayscale digital image(s) of a barcode marked or disposed on each surgical cassette during console setup as well as live images of a fluid level window within the surgical cassette before, during, and after surgery. Each image is processed as a matrix of numbers known as pixel brightness values. These pixel brightness values represent the intensity or brightness of each pixel in the 8-bit grayscale of 0 to 255, wherein a pixel brightness value of 0 represents “black,” and a pixel brightness value of 255 represents “white”. By using independent sets of internal light sources and capturing and processing the barcode and fluid level window images at separate times, the LSBR serves both functions of a fluid level sensor and a barcode reader in one integrated device.

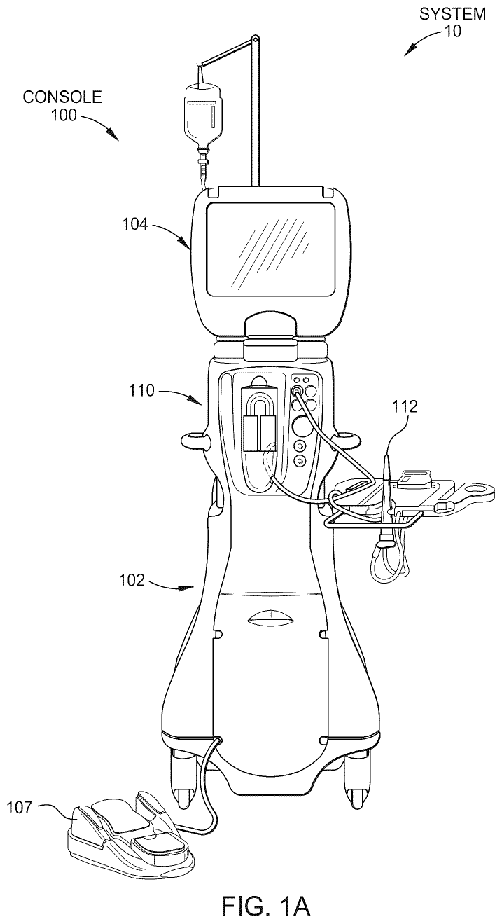

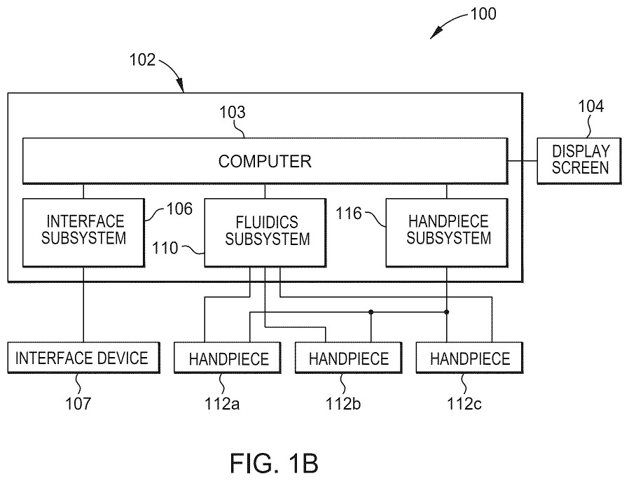

FIG. 1A illustrates an example of an ophthalmic surgical system 10 that may be used to perform ophthalmic procedures on an eye, according to certain embodiments. In the illustrated embodiments, system 10 includes console 100 (also referred to as a “surgical console”), a housing 102, a display screen 104, an interface device 107 (e.g., a foot pedal), a fluidics subsystem 110, and a handpiece 112, coupled as shown and described in more detail with reference to FIG. 1B.

FIG. 1B is an example of subsystems of console 100 of ophthalmic surgical system 10 of FIG. 1A, according to certain embodiments. Console 100 includes housing 102, which accommodates a computer 103 (with an associated display screen 104) and subsystems 106, 110, and 116, which support interface device 107 and handpieces 112 (112a-c). An interface device 107 receives input to surgical system 10, sends output from system 10, and/or processes the input and/or output. Examples of an interface device 107 include a foot pedal, manual input device (e.g., a keyboard), and a display. Interface subsystem 106 receives input from and/or sends output to interface device 107.

Handpiece 112 may be any suitable ophthalmic surgical instrument, e.g., an ultrasonically-driven phacoemulsification (phaco) handpiece, a laser handpiece, an irrigating cannula, a vitrectomy handpiece, or another suitable surgical handpiece. Fluidics subsystem 110 provides fluid control for one or more handpieces 112 (112a-c). For example, fluidics subsystem 110 may manage fluid for an irrigating cannula. Handpiece subsystem 116 supports one or more handpieces 112. For example, handpiece subsystem 116 may manage ultrasonic oscillation for a phaco handpiece, provide laser energy to a laser handpiece, control operation of an irrigating cannula, and/or manage features of a vitrectomy handpiece.

Computer 103 controls operation of ophthalmic surgical system 10. In certain embodiments, computer 103 includes a controller that sends instructions to components of system 10 to control system 10. A display screen 104 shows data provided by computer 103.

According to certain embodiments, computer 103 controls operation of ophthalmic surgical system 10. Generally, computer includes a processor and a memory. The memory may include any device operable to receive, store, or recall data, including, but not limited to, electronic, magnetic, or optical memory, whether volatile or non-volatile. The memory may include code stored thereon. The code may include instructions that may be executable by the processor. The code may be created, for example, using any programming language, including but not limited to, C++ or any other programming language (including assembly languages, hardware description languages, and database programming languages). In some instances, the code may be a program that, when loaded into the processor, causes the surgical console to receive and process information from one or more of subsystems 106, 110, and 116, for, e.g., providing fluid control for one or more handpieces 112.

The processor may be, or include, a microprocessor, a microcontroller, an embedded microcontroller, a programmable digital signal processor, or any other programmable device operable to receive information from the memory or other devices in communication with the processor, computer 103, and/or console 100, and perform one or more operations on the received information. For example, the processor may send instructions to components of fluidics subsystem 110, or other devices or systems in communication with computer 103, for controlling such devices and systems. The processor may also be operable to output results based on the operations performed thereby. A display screen 104 shows data and other output results provided by the processor of computer 103. In some instances, the processor may also be or include an application specific integrated circuit, a programmable gate array, programmable array logic, or any other device of combinations of devices operable to process electric signals.

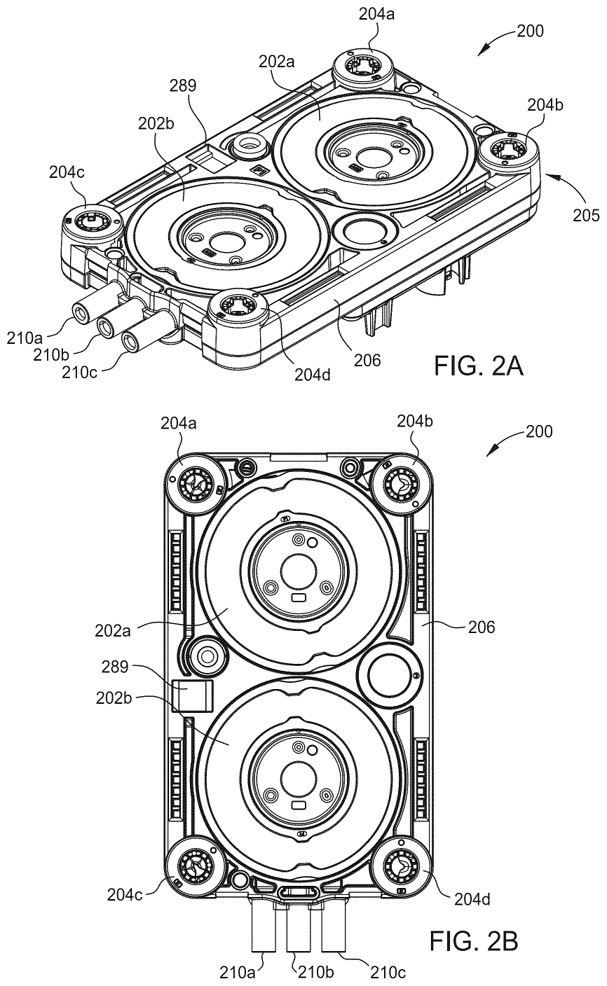

FIG. 2A is a back side isometric view of an example surgical cassette 200 which may be operably coupled to a console of an ophthalmic surgical system (e.g., console 100 of ophthalmic surgical system 10 illustrated in FIGS. 1A-1B), according to certain embodiments. FIG. 2B is a back side elevation view of surgical cassette 200 of FIG. 2A, according to certain embodiments. FIGS. 2A-2B are described together herein for clarity. Surgical cassette 200 includes two pump assemblies 202 (202a-b) which provide a source of pressure and/or vacuum and four valve assemblies 204 (204a-d) which control pressure and/or fluid communication within surgical cassette 200. In certain other embodiments, there may be only one pump assembly or more than two pump assemblies. In certain other embodiments, there may be more or less than four valve assemblies (e.g., two to six valve assemblies).

In certain embodiments, an external source of pressure and/or vacuum is coupled to surgical cassette 200. In such embodiments, the external source may be either in place of or in addition to pump assemblies 202.

Surgical cassette 200 has a housing 205 including a base 206, and inlet/outlet ports 210 (210a-c) in base 206 which provide pressure and/or fluid communication between inside and outside of the housing 205. Although not shown, flow lines (e.g., tubing) may be coupled between each port 210a-c and a corresponding component of fluidics subsystem 110 and/or a corresponding handpiece 112a-c (shown in FIGS. 1A-1B).

In certain embodiments, one of a first pump assembly 202a or second pump assembly 202b provides a source of pressure (e.g., to create a driving force for fluid irrigation) while the other one of the first pump assembly 202a or second pump assembly 202b provides a source of vacuum (e.g., to create suction for fluid aspiration). The first pump assembly 202a and second pump assembly 202b may be peristaltic pumps or any other suitable type of pump for generating pressure and/or vacuum. In certain embodiments, the first pump assembly 202a and second pump 202b assembly are identical to each other.

Valve assemblies 204 are coupled to base 206. Valve assemblies 204 function cooperatively to control pressure and/or fluid communication within and through surgical cassette 200. In the illustrated embodiments, surgical cassette 200 includes a first valve assembly 204a, a second valve assembly 204b, a third valve assembly 204c, and a fourth valve assembly 204d. As shown, in the embodiments of FIG. 2A, the four valve assemblies 204 are arranged at the four corners of housing 205 and surrounding the two pump assemblies 202 which are arranged towards a center of housing 205. However, in certain other embodiments, pump assemblies 202 and valve assemblies 204 may have any other suitable arrangement.

A venturi reservoir 282 is disposed inside housing 205 of surgical cassette 200. The Venturi reservoir 282 serves at least two primary functions that are integral to the operation of surgical cassette 200. In general, venturi reservoir 282 provides a connection to a vacuum source for suction of fluids during a venturi operation, provides a fluid volume sink for normal vacuum venting within the surgical cassette, and provides a fluid volume sink for venting vacuum pressure that may build-up within surgical cassette 200 in the event of a post-occlusion break surge. In certain embodiments, the venturi reservoir 282 also allows air to separate from liquid and then evacuate out the cassette 200 through the surgical console during normal aspiration/suction use with venturi vacuum.

Venturi reservoir 282 includes a barcode area 289 defined in base 206. Barcode area 289 is disposed along an optical path of an image sensor in console 100 that is used to decode a barcode and, according to certain embodiments, determine a fluid level in venturi reservoir 282. In certain embodiments, the image sensor is a single camera sensor, or a complementary metal-oxide semiconductor (CMOS) sensor. According to certain embodiments, the image sensor is configured to image an area that is 560 pixels by 560 pixels and has a nominal field of view of 12.32 mm by 12.32 mm, with a minimum field of view of 12 mm by 12 mm, and a maximum of 13 mm by 13 mm. During normal operation, a nominal fluid level in venturi reservoir 282 is between lower and upper limits of the fluid level window within the barcode area 289. The relatively small size or footprint of barcode area 289 enables the use of a more compact housing 205 compared to other designs.

In some embodiments, the surgical cassette 200 further comprises one or more features configured to interact with components of the surgical console 100 for facilitating identification of the type, serial number, pressure sensor calibration data or parameters, manufacturing date, batch number, etc., of surgical cassette 200 by the surgical console 100. In one embodiment, both features for facilitating identification of the surgical cassette 200 and measuring fluid levels within the venturi reservoir 282 disposed within the surgical cassette 200 are carried out by the LSBR of the surgical console 100.

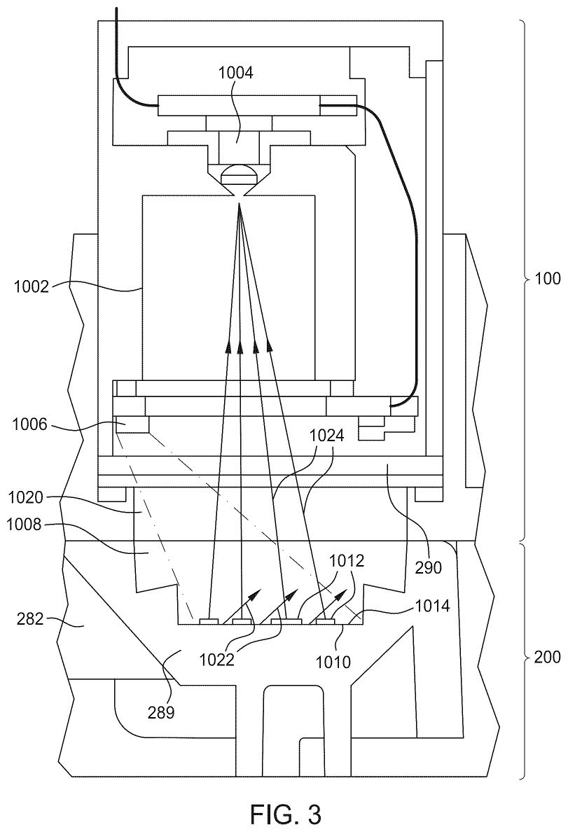

In certain embodiments, the features for facilitating identification comprises decoding a barcode 1016 disposed on the surgical cassette 200 as illustrated in FIG. 3, which shows a magnified top down cross sectional view of the interface between the barcode area 289 defined in the base 206 of the surgical cassette 200 and the LSBR 1002 disposed in the fluidics subsystem 110 of the surgical console 100. The LSBR 1002 comprises an image sensor 1004 and at least one visible light source 1006 that is configured or angled to emit visible light at an oblique angle relative to both a window 290 disposed in front of the LSBR 1002 within the surgical console 100 and to the barcode surface 1010 within the barcode area 289 of the surgical cassette 200. The barcode area 289 comprises a cavity 1008. The cavity 1008 comprises a barcode surface 1010 onto which a barcode 1016 is disposed. In one embodiment, the barcode 1016 is laser-etched onto the barcode surface 1010, namely with the barcode 1016 comprising a plurality of smooth surfaces 1014 intermingled or interspaced with a plurality of etched surfaces 1012 created by the laser. In one embodiment, the smooth surfaces 1014 of the barcode 1016 are comprised of smooth, dark, black, or other light absorbing plastic, however because the etched surfaces 1012 of the barcode 1016 have had their topmost layer roughened by a laser, each of the etched surfaces 1012 comprises a rough or uneven surface that can diffuse or scatter light.

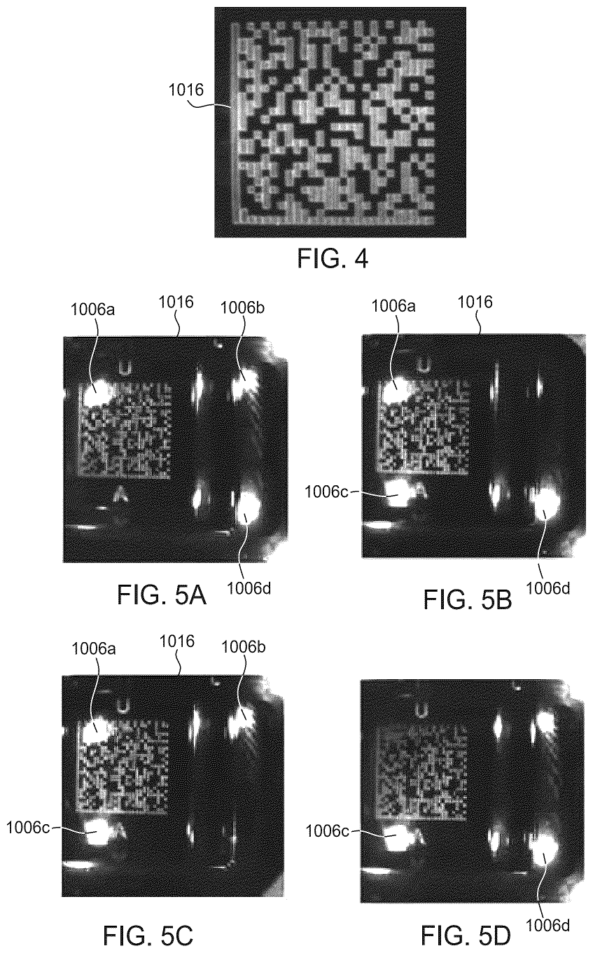

In one embodiment, for the surgical console 100 to decode the barcode 1016 disposed within the surgical cassette 200, visible light 1020 is emitted from the at least one visible light source 1006 along an optical path that enters the cavity 1008 via the window 290 disposed within the surgical console 100 at an oblique angle relative to the window 290. After being transmitted through the window 290, the visible light 1020 strikes the barcode 1016 on the barcode surface 1010, the visible light 1020 illuminating both the smooth surfaces 1014 and the etched surfaces 1012 of the barcode 1016 at an oblique angle. The portions of the visible light 1020 which illuminate the smooth surfaces 1014 of the barcode 1016 are either reflected away from the image sensor 1004 as indicated by arrows 1022 or are absorbed by the material comprising the barcode surface 1010, while the portions of the visible light 1020 that illuminate the etched surfaces 1012 are diffused, some of which are directed back through the window 290 and detected by the image sensor 1004 as indicated by lines 1024. The end result, as seen in FIG. 4, is an image of the barcode 1016 comprised of light and dark portions, the light portions representing the light 1024 that was directed back at the image sensor 1004 by the etched surfaces 1012, and the dark portions representing the light 1022 which was reflected away or absorbed by the smooth surfaces 1014. Therefore under proper lighting conditions, the surgical console 100 using known software can then decode the image of the barcode 1016 and thereby obtain stored data/information of surgical cassette 200 currently inserted into the fluidics subsystem 110 of the surgical console 100 including pressure sensors calibration data/parameters, cassette identification, and manufacturing date, which ensures that the cassette 200 is the correct cassette and that the console 100 can function correctly for the procedure currently being performed on a patient.

In one embodiment, the LSBR 1002 comprises four different visible light sources each of which are configured to illuminate the barcode 1016 at an oblique angle as disclosed above. According to certain embodiments, each light source provides light with a wavelength of 630 nm. As seen in FIGS. 5A-5D, references 1006a-d correspond to the light reflected off of barcode surface 1010 from each of the four different visible light sources, respectively. In a related embodiment, the four visible light sources are symmetrically disposed about the image sensor 1004, namely with at least two visible light sources disposed on each lateral side of the image sensor 1004, thereby disposing the image sensor 1004 in the middle or center of the disposed visible light sources. However when multiple visible light sources are used and an image is acquired by the image sensor 1004, the glare or amount of light that is reflected from each of the visible light sources 1006a-d can obfuscate the barcode 1016 and render it difficult for the user to detect. To solve this problem, the image sensor 1004 captures a sequence of images of the barcode 1016 with each image within the sequence having at least one of the visible light sources 1006a-d darkened or turned off while the remaining visible light sources 1006a-d illuminated. For example, as seen in FIGS. 5A-5D four images are acquired by the image sensor 1004 with each one of the images comprising a different one of the plurality of visible light sources darkened or turned off, thereby producing a corresponding number of reflections 1006a-d. FIG. 5A is an illustration of a first image taken within the sequence in which a third visible light source is darkened, while a first visible light source, a second visible light source, and a fourth visible light source remain illuminated, thereby producing a corresponding first reflection 1006a, a second reflection 1006b, and a fourth reflection 1006d.

As seen in FIG. 5A, the light reflected from the illuminated visible light sources blocks or partially blocks the corresponding portions of the barcode 1016, while the portion of the barcode 1016 corresponding to where the third visible light source is disposed remains dark, thereby allowing the diffused light 1024 from the etched surfaces 1012 to clearly appear within the image. The process is then repeated for each of the remaining visible light sources, namely with FIG. 5B illustrating how the barcode 1016 appears when the second visible light source is darkened, FIG. 5C illustrating how the barcode 1016 appears when the fourth visible light source is darkened, and FIG. 5D illustrating how the barcode 1016 appears when the first visible light source is darkened.

After all images of the barcode 1016 are obtained, image processing software within the surgical console 100 combines all of the images seen in FIGS. 5A-5D to create a single image of the barcode 1016 as shown in FIG. 4 by selectively combining the darkened portions of each respective image of FIGS. 5A-5D into a single composite image as seen in FIG. 4 which comprises the most clearly received diffused light 1024 from each portion of the barcode 1016. While four visible light sources 1006a-d and four images are seen in FIGS. 5A-5D, it is to be expressly understood that fewer or additional images may be taken or that fewer or additional visible light sources may be used other than what is explicitly disclosed herein. For example, in one particular embodiment, the image sensor 1004 may only take two images, a first image where only the first and second visible light sources 1006a, 1006b are illuminated, and a second image where only the third and fourth visible light sources 1006c, 1006d are illuminated. The internal software within the surgical console 100 may then overlay or “stitch” the bottom portion of the first image to the top portion of the second image to create a single final image which comprises the clearest or most readable portions of the barcode 1016. In certain embodiments, a final image is provided consisting of three regions, namely a top region, a middle region, and a bottom region. In some embodiments, the bottom region will be from a first image, the top region will be from a second image, and the middle region will be a transition zone where the middle region comprises of a blend of the first and second images. At a bottom edge of the middle region, this could, for example, be 100% from the first image and 0% from the second image which linearly transitions to 0% from the first image and 100% from the second image at a top edge of the middle region.

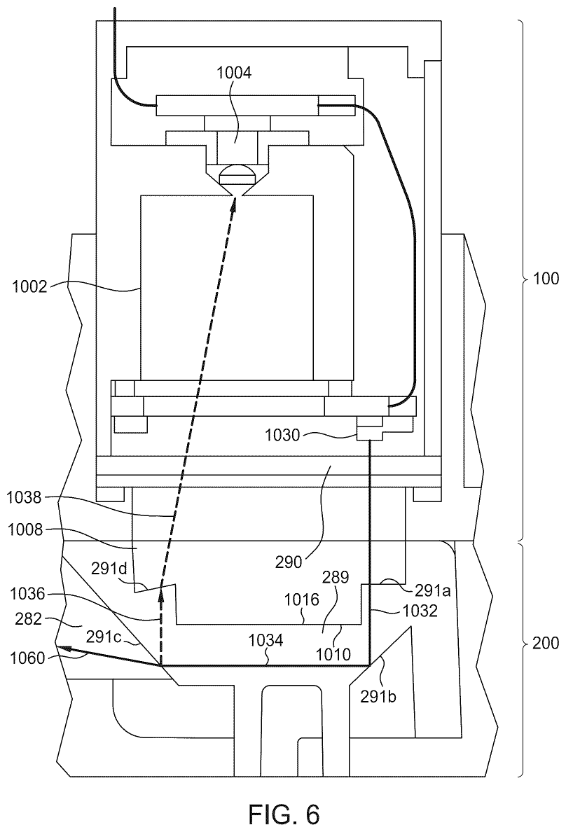

In some embodiments, the features for determining a current fluid level within the venturi reservoir 282 disposed within the surgical cassette 200 is illustrated in FIG. 6. As seen in FIG. 6, venturi reservoir 282 is adjacent the barcode area 289 defined in base 206. When the surgical cassette 200 is coupled to the surgical console 100, barcode area 289 is disposed along an optical path of the image sensor 1004 in surgical console 100 that is used to determine a fluid level in venturi reservoir 282.

The cavity 1008 of the barcode area 289 seen in FIG. 6 comprises a plurality of internal surfaces 291a-d, which in some embodiments includes a normal surface 291a and angled surfaces 291b, 291c, and 291d. In certain embodiments, the plurality of internal surfaces 291a-d may comprise any combination of flat, angled, or curved configurations while maintaining the same functionality. For example, in one embodiment, the surface 291a is flat and normal to the optical path of incoming infrared light emitted by an infrared light source 1030 disposed within LSBR 1002 of the surgical console 100, indicated by arrow 1032. After being transmitted through the surface 291a, the infrared (IR) light contacts the first angled surface 291b which is aligned with both the infrared light source 1030 in LSBR 1002 of console 100 and the venturi reservoir 282. The first angled surface 291b comprises a polish or texture, as well as an angular orientation (if flat) or curvature, to help direct the IR light from the IR light source 1030 towards the second angled surface 291c. In other words, IR light that is emitted from LSBR 1002 of console 100 reflects off the first angled surface 291b, passes from the viewer's right to left along the optical path as indicated by arrow 1034, then partially reflects off the second angled surface 291c (which is flat or curved) away from the viewer back towards LSBR 1002 of console 100 along the optical path as indicated by the dashed line arrow 1036. The portion of infrared light that is reflected back towards LSBR 1002 of console 100 passes through the third angled surface 291d which is orientated to redirect the reflected IR light through the window 290 disposed within LSBR 1002 of the surgical console 100 and onto the image sensor 1004 of LSBR 1002 in surgical console 100 for detection along the optical path as indicated by dashed line arrow 1038. In some embodiments, each angled surface 291b, 291c is oriented at about 45 degrees in relation to a plane of base 206 so that the infrared light reflected back towards LSBR 1002 in console 100 is reversed, or rotated by about 180 degrees, in relation to the infrared light emitted from LSBR 1002 in console 100. However, in some other embodiments, angled surfaces 291b, 291c and angled surface 291d are oriented at other angles that correspond to the positions and/or orientations of the infrared light source 1030 and image sensor 1004.

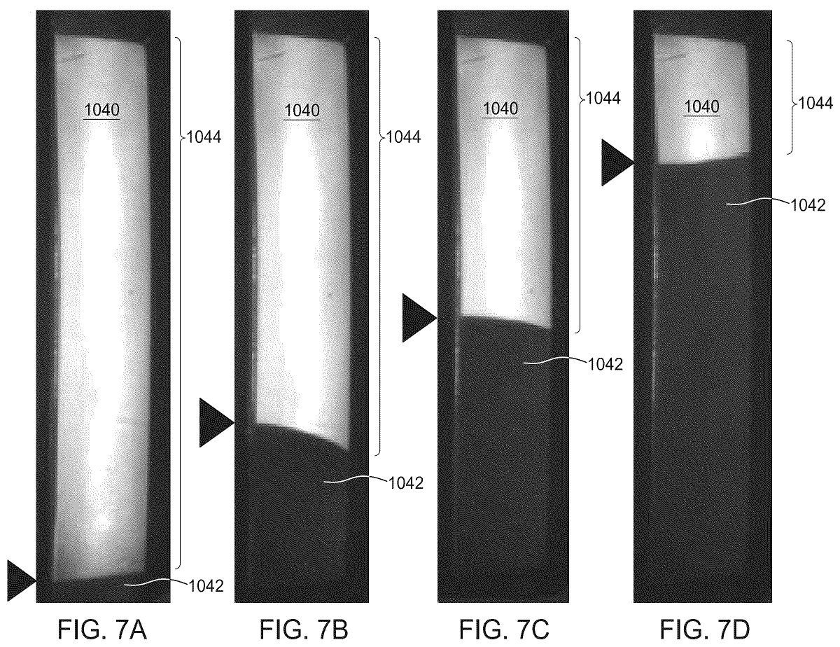

Detection of the relative fluid level within the venturi reservoir 282 works based on the difference in index of refraction of air compared to liquid at surface 291c. In one embodiment, as IR light strikes the second angled surface 291c, a first portion of the IR light that is incident where there is liquid present on surface 291c, is refracted at the solid-liquid interface and continues along path 1060 into the venturi reservoir 282, while a second or remaining portion of the IR light, incident where there is no liquid present on surface 291c, undergoes total internal reflection off of the second angled surface 291c and is then directed to the angled surface 291d. Following this process, an image of surface 291c is projected into the image sensor 1004 of LSBR 1002. Regions of the surface that are in contact with liquid appear dark, regions that are in contact with air appear light. This image can be analyzed to determine the fluid level. The proportion or amount of IR light which enters the fluid to the proportion or amount of IR light 1036 which undergoes total internal reflection and is reflected off of the second angled surface 291c depends upon the amount of fluid currently contained within the venturi reservoir 282. Specifically, the portion of incoming IR light 1034 which strikes a corresponding surface portion of the venturi reservoir 282 which has fluid disposed behind it will refract into the venturi reservoir 282 and thus not ever be detected by the image sensor 1004. Meanwhile, the portion of incoming IR light 1034 which strikes a corresponding surface portion of the venturi reservoir 282 which does not have fluid behind it will instead undergo total internal reflection and reflect off of the second angled surface 291c and then subsequently detected by the image sensor 1004 of LSBR 1002. The net effect therefore is that IR light is absorbed where there is fluid in the venturi reservoir 282 and reflected where it is not, thereby providing a means to determine the exact current amount of fluid within the venturi reservoir 282. An image of surface 291c is directed by surface 291d and projected into the image sensor 1004 of LSBR 1002 in the shape and size of surface 291d, which is defined as fluid level window 1044 as seen in FIG. 7. In certain embodiments, the fluid level window 1044 is part of the barcode area 289 being seen by the image sensor 1004 of LSBR 1002, namely both the barcode 1016 and the fluid level window 1044 are within the field of view of the image sensor 1004 of LSBR 1002.

FIGS. 7A-D illustrate a series of example images of fluid level windows 1044 captured by the image sensor 1004 of LSBR 1002 as a part of barcode area 289 when detecting multiple different fluid levels within the venturi reservoir 282. As seen in FIG. 7A, a majority of IR light has been detected by the image sensor 1004, thereby providing a reading of fluid level with a light portion 1040 occupying the majority of a fluid level window 1044 (corresponding to the amount of light reflected from second angled surface 291c) and indicating that the venturi reservoir 282 is empty or nearly empty. In FIG. 7B, a large portion of IR light has been detected by the image sensor 1004, thereby providing a reading of fluid level with a light portion 1040 occupying a top ¾ of the fluid level window 1044 and a dark portion 1042 occupying the remaining bottom ¼ of the fluid level window 1044 and indicating that the venturi reservoir 282 is approximately ¼ full of fluid. In FIG. 7C, a portion of IR light has been detected by the image sensor 1004, thereby providing a reading of fluid level with a light portion 1040 occupying a top ½ of the fluid level window 1044 and a dark portion 1042 occupying the remaining bottom ½ of the fluid level window 1044 and indicating that the venturi reservoir 282 is approximately ½ full of fluid. In FIG. 7D, only a small portion of IR light has been detected by the image sensor 1004, thereby providing a reading of fluid level with a light portion 1040 occupying a top ¼ of the fluid level window 1044 and a dark portion 1042 occupying the remaining bottom ¾ of the fluid level window 1044 and indicating that the venturi reservoir 282 is approximately ¾ full of fluid.

Problems can develop however when sources of external or ambient light are introduced during either the barcode decoding procedure seen in FIG. 3 or the fluid level sensing procedure seen in FIG. 6. That is because any external light seen by the image sensor 1004 of LSBR 1002 is noise which may interfere with the barcode 1016 and or the fluid level window 1044. For blocking external light, making the cassette from opaque material(s), or covering the entire front surfaces of the cassette with opaque films may be effective. However, due to manufacturability and inspection considerations, the surgical cassette 200, according to certain embodiments, is made of transparent or translucent material(s) and only a small area directly in front of barcode area 289 is covered with an opaque film. Therefore, when a strong external light source is directly pointed toward the surgical cassette 200, such light may enter the surgical cassette 200 through areas and structures not covered by a light blocking film or other light mitigation means and bounce around internally, which could bring the leakage of light to a high enough level as to cause interference with proper functions of the image sensor 1004. For example, in the case of barcode reader, a strong external light may significantly reduce the barcode pattern contrast or obscure the barcode pattern image rendering the barcode 1016 undecodable. The data and information stored in the barcode 1016 of each surgical cassette 200 includes pressure sensors calibration data and parameters for each cassette type. In short, the surgical console 100 simply cannot work without decoding the barcode 1016. In the case of the level sensor, a strong external light may significantly increase the brightness within the fluid level window 1044 or appear as bright spots in the fluid-filled region of the dark portion 1042, which is intended to be dark. Proper fluid level determination requires adequate image contrast, namely a large enough difference between the brightness of the air region versus the darkness of the fluid region. Insufficient image contrast may cause erroneous fluid level readings, which when fed back to the fluidics subsystem 110, may cause the true fluid level to rise or fall in a manner that renders the fluidics subsystem 110 inoperable.

The current invention provides a method for utilizing the image sensor 1004 to acquire grayscale image(s) and the computer 103 to then process the image(s) to decode the barcode 1016 and/or determine the current level of fluid within the surgical cassette 200. The image sensor 1004 is further utilized to detect external light that comes into the field of view of the image sensor 1004. A software algorithm stored within the computer 103 in the surgical console 100 determines whether the potential is high for external light interference. If there is a high potential for external light interference, an advisory or alarm is generated to warn the user to remove the external light source or to turn the console away from the external light source. Depending on the risk level, for example during surgery or setup, certain operations or functions of the surgical console 100 may also then be suspended. For example, the surgical console 100 may enter a safe state, or “safe mode,” until the interference is eliminated. Once the user has removed the external light and then cleared the advisory, normal console operations and functions can resume.

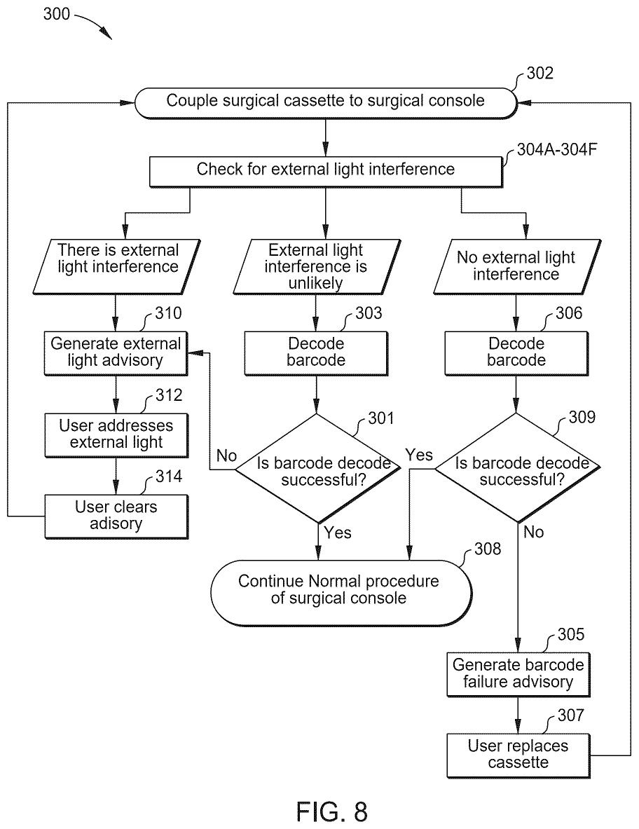

According to certain embodiments, the method of checking external light interference of the current invention is incorporated into a procedure 300 for coupling and connecting the surgical cassette 200 to the fluidics subsystem 110 of the surgical console 100, as seen in the flow chart of FIG. 8. The procedure begins with step 302, where a user couples the surgical cassette 200 to the fluidics subsystem 110 of the surgical console 100, which brings the barcode area 289 into alignment with window 290 of LSBR 1002 in the surgical console 100. In step 304A-F, the image sensor 1004 is activated in order to perform a test for determining if any external light may be inadvertently shining on the surgical cassette 200 and interfering with decoding of the barcode 1016 and/or fluid level sensing. Step 304A-F determines if external light interference is present, if external light interference is unlikely, or if there is no external light interference.

If external light interference is confirmed, console 100 will generate an external light advisory in step 310 to direct the user to remove the external light source or to turn the console 100 away from the external light source. After the user addresses the external light in step 312 and clears the advisory in step 314, the procedure returns to step 304A-F.

If it is determined that there is no external light interference, the console moves forward to barcode decoding step 306. Subsequently, if decoding barcode is successful during step 309, normal console operating procedure continues in step 308, otherwise the console 100 generates barcode failure advisory in step 305 to direct the user to replace the cassette 200. After user replaces the cassette 200 in step 307, the procedure returns to step 302, where a new surgical cassette 200 is coupled to the fluidics subsystem 110 of the surgical console 100.

If external light interference is determined to be unlikely, the console 100 moves forward to barcode decoding in step 303. Subsequently, if decoding barcode is successful during step 301, normal console operating procedure continues in step 308, otherwise console generates external light advisory in step 310 to direct the user to remove the external light source or turn the console away from the external light source. After user addresses the external light in step 312 and clears the advisory in step 314, the procedure returns to step 304A-F.

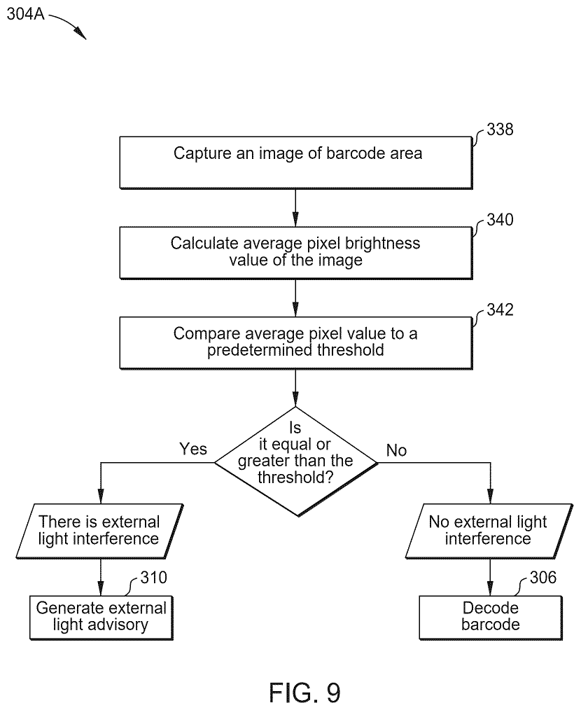

According to certain embodiments as seen in FIG. 9, step 304A comprises the image sensor 1004 capturing a grayscale image of the barcode area 289 of the surgical cassette 200 in step 338 using a default or predetermined exposure, gain, and frame rate, while the internal visible light sources 1006a-d and the infrared light source 1030 remain off. According to certain embodiments, the image sensor 1004 has a default exposure of 0.5 ms, a gain of 3.0×, and a frame rate of 60 frames per second. Next, the computer 103 within the surgical console 100 processes and records the intensity or brightness value for each pixel of the grayscale image in step, namely with each pixel having a recorded value between zero, or black, and 255, or white. The computer 103 then calculates, in step 340, an average pixel brightness value for the entire grayscale image and then compares that calculated value to a predetermined threshold value stored within the computer 103 in step 342. If the calculated average value is equal to or greater than the threshold value, external light interference has been detected and an advisory alarm (or advisory notice), alert, image, or message is sent to the user in step 310 as seen in FIG. 8, (e.g., via display screen 104 of the surgical console 100). The user then progresses through the subsequent procedure steps illustrated in FIG. 8. Alternatively, if the calculated average value is less than the predetermined threshold, then no external light interference has been detected and the image sensor 1004 is then permitted to decode the barcode 1016 in step 306 as seen in FIG. 8. The user then progresses through the subsequent procedure steps illustrated in FIG. 8.

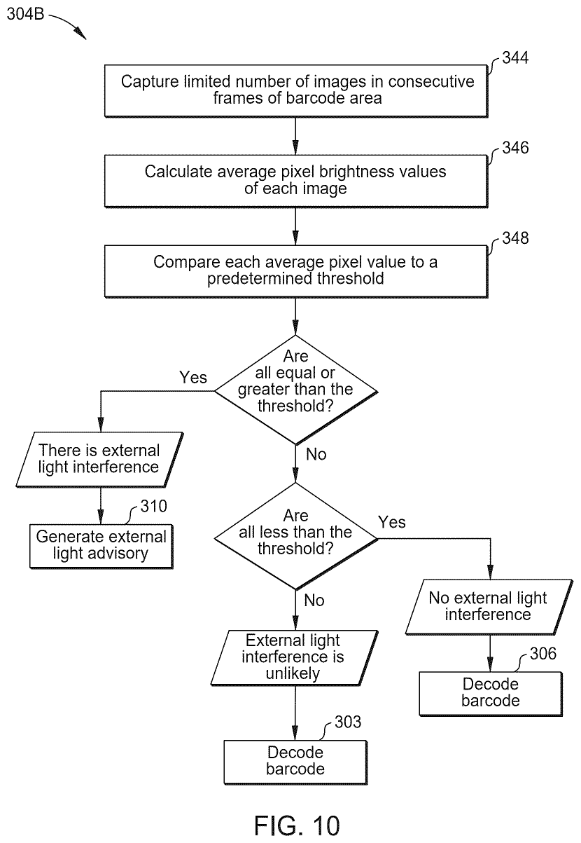

According to certain embodiments as seen in FIG. 10, step 304B comprises the image sensor 1004 capturing a limited plurality of grayscale images in consecutive frames of the barcode area 289 of the surgical cassette 200 in step 344. For example, according to one embodiment, the image sensor 1004 captures a sequence of sixteen (16) consecutive frames. The specific number of consecutive images represents the consistent existence of an external light condition for its duration. For example, 16 frames divided by 60 frames per second represents a total time of 0.267 seconds. Comparing to the single image method of FIG. 9, using a limited number of images to check for external light interference can filter out transitory conditions that may be detected over a single image. The computer 103 within the surgical console 100 calculates an average pixel brightness value for each image in step 346, and then compares each of the calculated average pixel brightness values to a predetermined threshold value stored within the computer 103 in step 348. If all of the calculated average values are equal to or greater than the threshold value, external light interference has been detected and an advisory alarm (or advisory notice), alert, image, or message is sent to the user in step 310 as seen in FIG. 8. The user then progress through the subsequent procedure steps illustrated in FIG. 8. Alternatively, if all of the calculated average values are less than the predetermined threshold, then no external light interference has been detected and the image sensor 1004 is then permitted to decode the barcode 1016 in step 306 as seen in FIG. 8. The user then progresses through the subsequent procedure steps illustrated in FIG. 8. If the calculated average pixel brightness values for some, but not all, are equal to or greater than the predetermined threshold, external light interference is unlikely and the image sensor 1004 is allowed to decode the barcode 1016 in step 303 as seen in FIG. 8. The user then progresses through the subsequent procedure steps illustrated in FIG. 8.

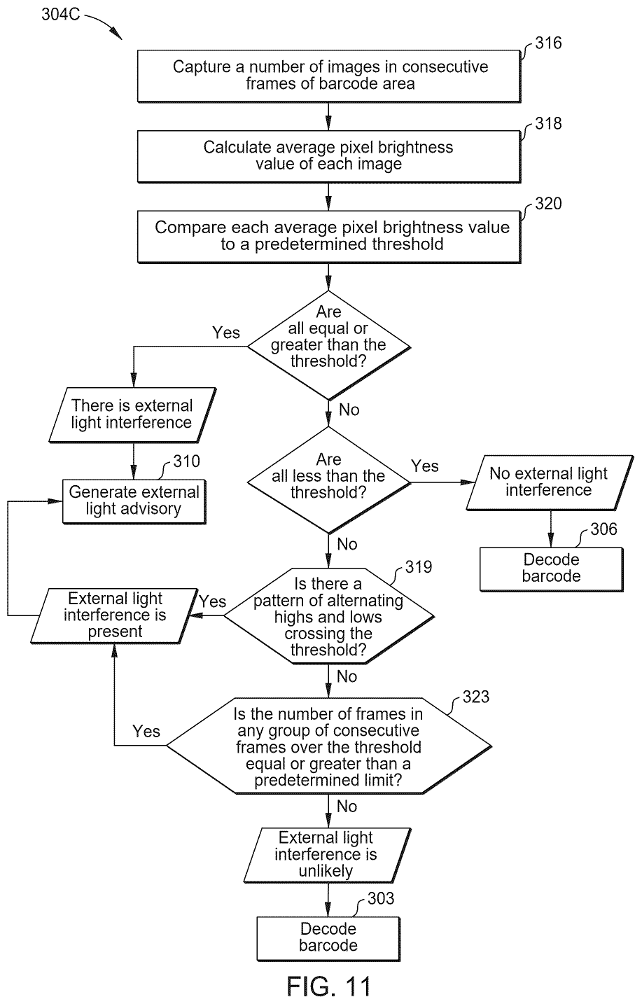

According to certain embodiments as seen in FIG. 11, step 304C comprises the image sensor 1004 capturing a large plurality of grayscale images in consecutive frames of the barcode area 289 of the surgical cassette 200 in step 316. For example, according to one embodiment, the image sensor 1004 captures a sequence of 120 consecutive frames. The specific number of consecutive images represents the permanent or periodic existence of an external light condition for its duration. For example, 120 frames divided by 60 frames per second represents a total time of 2 seconds. Comparing to the single image method of FIG. 9 and the limited number of images method in FIG. 10, using a large number of images to check for external light interference is more likely to detect external light conditions that are short pulses but are cyclical over an extended period of time and thus unlikely to be detected within shorter analysis periods, for example IR remotes which have a certain on and off frequency. The computer 103 calculates an average pixel brightness value for each of the plurality of grayscale images in step 318, and then compares each of the calculated average pixel brightness values to a predetermined threshold value stored within the computer 103 in step 320. If all of the calculated average values are equal to or greater than the threshold value, external light interference has been detected and an advisory alarm (or advisory notice), alert, image, or message is sent to the user in step 310 as seen FIG. 8. The user then progresses through the subsequent procedure steps illustrated in FIG. 8. Alternatively, if all of the calculated average values are less than the predetermined threshold, then no external light interference has been detected, and the image sensor 1004 is then permitted to decode the barcode 1016 in step 306 as seen in FIG. 8. The user then progresses through the subsequent procedure steps illustrated in FIG. 8.

According to certain embodiments, if the calculated average values for some, but not all, are equal to or greater than the predetermined threshold, the computer 103 then determines whether or not there is a repeating or cyclical pattern of the calculated average values repeatedly crossing the predetermined threshold in step 319. If there is an alternating pattern of calculated average values repeatedly crossing the predetermined threshold, external light interference is present, and an advisory is generated for the user in step 310 as seen in FIG. 8. The user then progresses through the subsequent procedure steps illustrated in FIG. 8. If there is no pattern of calculated average values repeatedly crossing the predetermined threshold, according to certain embodiments, the computer 103 then counts the number of frames in every group of consecutive frames that have an average pixel brightness value equal to or greater than the predetermined threshold and compares the frame number counts against a predetermined limit, for example 16 frames, in step 323. If at least one such group's frame number count is equal to or larger than the predetermined limit, external light interference is present, and the advisory is generated in step 310 as seen in FIG. 8. The user then progresses through the subsequent procedure steps illustrated in FIG. 8. If no such group's frame number count is equal to or larger than the predetermined limit, external light interference is unlikely, and the surgical console 100 is allowed to proceed forward to barcode decoding in step 303 as seen in FIG. 8. The user then progresses through the subsequent procedure steps illustrated in FIG. 8.

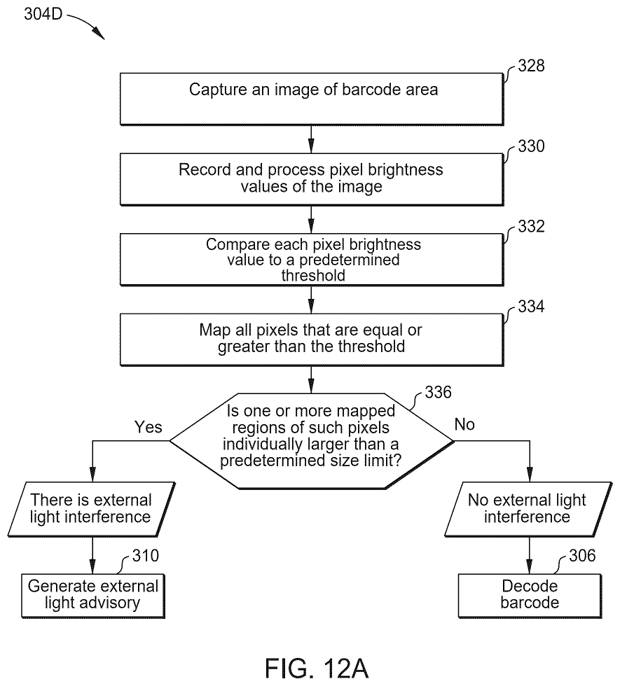

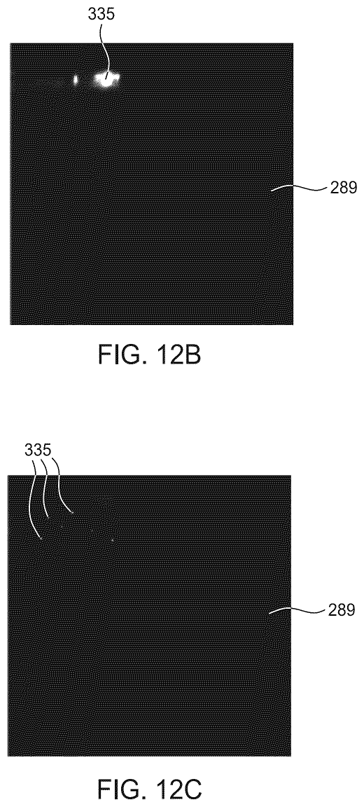

According to certain embodiments, step 304D as seen in FIG. 12A comprises the image sensor 1004 capturing a grayscale image of the barcode area 289 of the surgical cassette 200 in step 328. The computer 103 within the surgical console 100 records and processes the intensity or brightness values for each pixel of the image in step 330. The computer 103 then compares the brightness values of each pixel to a predetermined threshold value stored within the computer 103 in step 332. According to certain embodiments, the predetermined threshold value is 10. Next, according to certain embodiments, a map of all pixels which have been determined to have a brightness value equal to or greater than the predetermined threshold is generated in step 334. The computer 103 then determines if the generated map contains any regions therein that are individually larger than a predetermined size limit in step 336. For example, as seen in FIG. 12B, an exemplary image of the barcode area 289 is shown wherein a local bright region 335 of pixels that have been determined to have a pixel brightness value greater than the predetermined threshold and an area or size larger than a predetermined size limit. According to certain embodiments, the predetermined size limit is a region size that is 5 pixels by 5 pixels. Small spots of external light, for example bright regions 335 that are equal to or less than 5 pixels by 5 pixels, as seen in FIG. 12C, do not impact barcode decoding since the decoding program allows for a predetermined level of barcode pattern distortion or damage. Small spots of external light have no real effect on fluid level sensing either because the level sensor algorithm is designed to be robust enough in dealing with low level noises such as small air bubbles which are similar to small spots of external light.

By mapping regions of external light which are determined to be above the size threshold, the current method detects local external light spot(s) that could interfere with the proper functions of the image sensor 1004 which would otherwise be missed when averaging the pixel brightness values over the whole image since by averaging, an external light spot of limited size does not have much effect on the overall average pixel brightness value of the whole image. If at least one region within the generated map is individually larger than the predetermined size limit, external light interference has been detected and an advisory alarm (or advisory notice), alert, image, or message is generated for the user in step 310 as seen in FIG. 8. The user then progresses through the subsequent procedure steps illustrated in FIG. 8. Alternatively, if no region within the generated map is individually larger than the predetermined size limit, then no external light interference has been detected, and the image sensor 1004 is then permitted to decode the barcode 1016 in step 306 as seen in FIG. 8. The user then progresses through the subsequent procedure steps illustrated in FIG. 8.

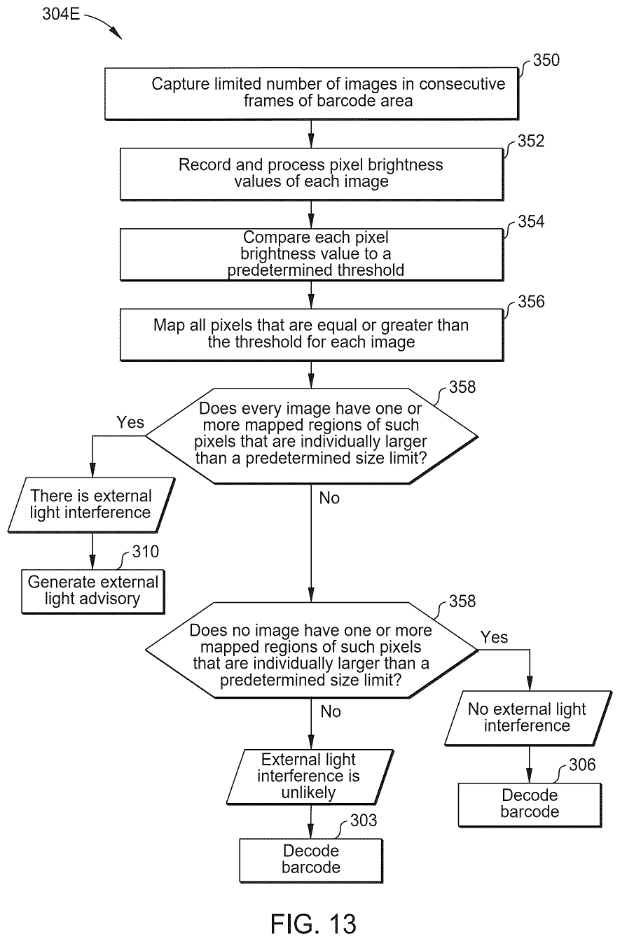

In one embodiment, step 304E as seen in FIG. 13 comprises the image sensor 1004 capturing a limited plurality of grayscale images of the barcode area 289 of the surgical cassette 200 in step 350. For example, according to one embodiment, the image sensor 1004 captures a sequence of sixteen (16) consecutive frames. The specific number of consecutive images represents the consistent existence of an external light condition for its duration. For example, 16 frames divided by 60 frames per second represents a total time of 0.267 seconds. The computer 103 within the surgical console 100 records and processes the intensity or brightness for each pixel in each of the limited plurality of images in step 352. The computer 103 then compares the brightness values of each pixel within each image to a predetermined threshold value stored within the computer 103 in step 354. According to certain embodiments, the predetermined threshold value is 10.

Next, according to certain embodiments, a map of all pixels which have been determined to have a brightness value equal to or greater than the predetermined threshold is generated in step 356 for each of the limited plurality of images. The computer 103 then determines if the generated map for each image contains any regions therein that are individually larger than a predetermined size limit in step 358. According to certain embodiments, the predetermined size limit is a region size that is 5 pixels by 5 pixels. If every image contains at least one mapped region that is individually larger than the predetermined size limit, external light interference has been detected and an advisory alarm (or advisory notice), alert, image, or message is generated for the user in step 310 as seen in FIG. 8. The user then progresses through the subsequent procedure steps illustrated in FIG. 8. Alternatively, if no image contains at least one mapped region that is individually larger than the predetermined size limit, then no external light interference has been detected and the image sensor 1004 is then permitted to decode the barcode 1016 in step 306 as seen in FIG. 8. The user then progresses through the subsequent procedure steps illustrated in FIG. 8. If some, but not all, of the images have at least one mapped region that is individually larger than the predetermined size limit, external light interference is unlikely and the image sensor 1004 is allowed to decode the barcode 1016 in step 303 as seen in FIG. 8. The user then progresses through the subsequent procedure steps illustrated in FIG. 8.

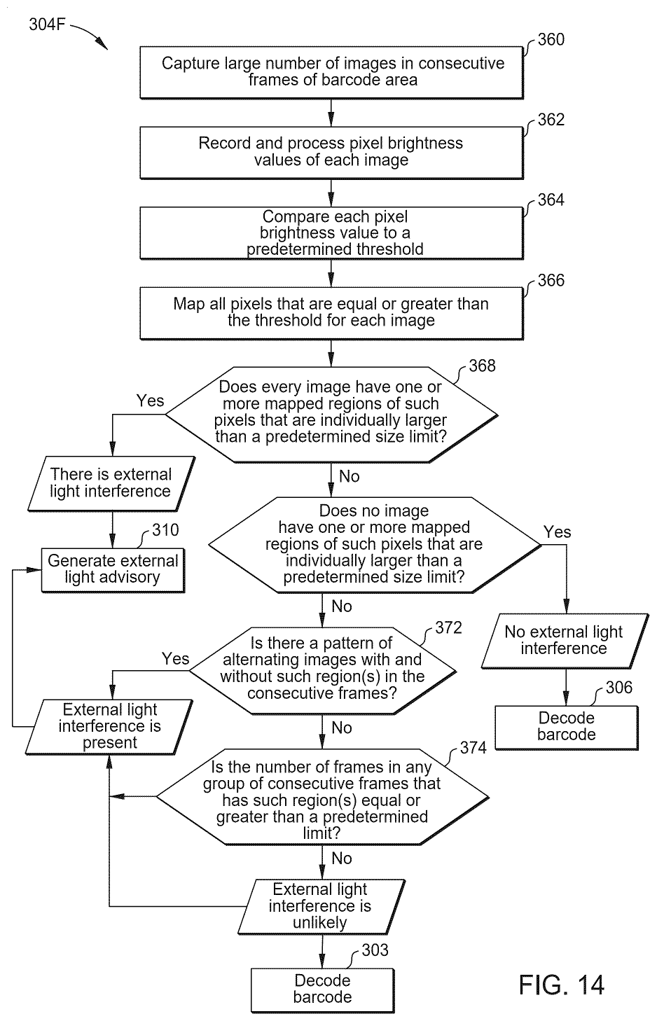

In one embodiment as seen in FIG. 14, step 304F comprises the image sensor 1004 capturing a large plurality of grayscale images in consecutive frames of the barcode area 289 of the surgical cassette 200 in step 360. For example, according to one embodiment, the image sensor 1004 captures a sequence of 120 consecutive frames. The specific number of consecutive images represents the permanent or periodic existence of an external light condition for its duration. For example, 120 frames divided by 60 frames per second represents a total time of 2 seconds. Comparing to the single image method of FIGS. 9 and 12A and the limited plurality of images method in FIGS. 10 and 13, using a large number of images to test or check for external light interference is more likely to detect external light conditions that are short pulses but are cyclical over an extended period of time and thus unlikely to be detected within shorter analysis periods. The computer 103 within the surgical console 100 records and processes the intensity or brightness values for each pixel in each of the large plurality of grayscale images in step 362. The computer 103 then compares each pixel brightness value to a predetermined threshold value stored within the computer 103 in step 364. According to certain embodiments, the predetermined threshold value is 10.

Next, according to certain embodiments, a map of all pixels which have been determined to have a brightness value equal to or greater than the predetermined threshold is generated in step 366 for each of the large plurality of images. The computer 103 then determines if the generated map for each image contains any regions therein that are individually larger than a predetermined size limit in step 368. According to certain embodiments, the predetermined size limit is a region size that is at least 5 pixels by 5 pixels. If every image contains at least one mapped region that is individually larger than the predetermined size limit, external light interference has been detected and an advisory alarm (or advisory notice), alert, image, or message is generated for the user in step 310 as seen in FIG. 8. The user then progresses through the subsequent procedure steps illustrated in FIG. 8. Alternatively, if no image contains at least one mapped region that is individually larger than the predetermined size limit, then no external light interference has been detected and the image sensor 1004 is then permitted to decode the barcode 1016 in step 306 as seen in FIG. 8. The user then progresses through the subsequent procedure steps illustrated in FIG. 8.

According to certain embodiments, if some, but not all, of the large plurality of images comprise at least one mapped region that is individually larger than the predetermined size limit, the computer 103 then determines whether or not there is a repeating or cyclical pattern of alternating images that are with and without at least one mapped region individually larger than the predetermined size limit in the large number of consecutive frames in step 372. If there is such pattern then external light interference is present, and an advisory is sent to the user in step 310 as seen in FIG. 8. The user then progresses through the subsequent procedure steps illustrated in FIG. 8. If there is no such pattern, according to certain embodiments, the computer 103 then counts the number of frames in every group of consecutive frames that have at least one mapped region that is individually larger than the predetermined size limit in step 374. If at least one such group's frame number count is equal to or greater than a predetermined limit, for example 16, external light interference is present, and an advisory is sent to the user in step 310 as seen in FIG. 8. The user then progresses through the subsequent procedure steps illustrated in FIG. 8. If, however, one such group's frame number count is less than the predetermined limit, external light is unlikely to be present and the image sensor 1004 is allowed to decode the barcode 1016 in step 303 as seen in FIG. 8. The user then progresses through the subsequent procedure steps illustrated in FIG. 8.

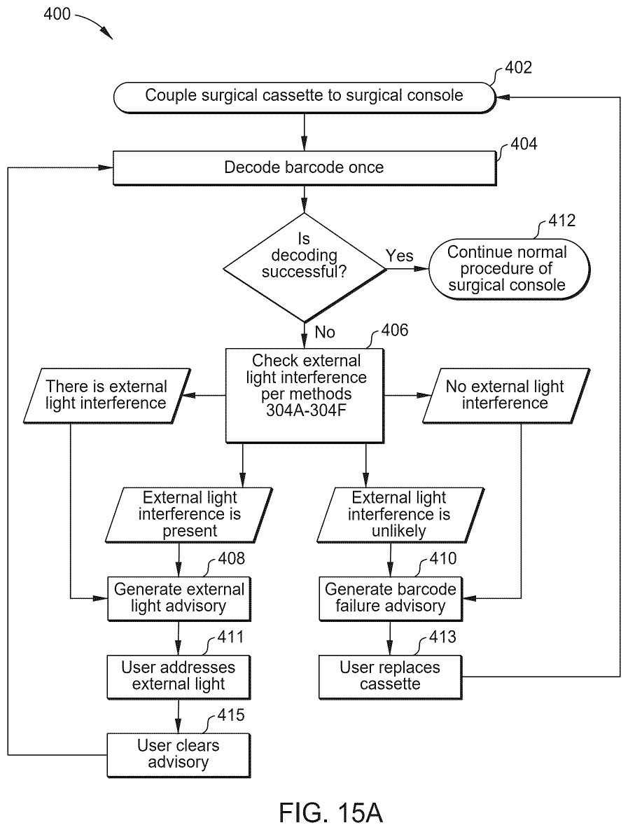

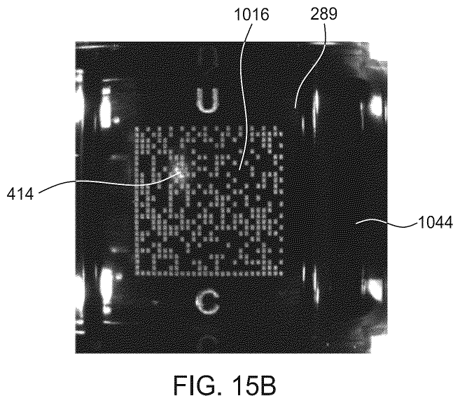

According to certain embodiments, the method of checking external light interference of the current invention is incorporated into a procedure 400 for coupling and connecting the surgical cassette 200 to the fluidics subsystem 110 of the surgical console 100, as seen in the flow chart of FIG. 15A. The procedure 400 begins with step 402, where a user couples the surgical cassette 200 to the fluidics subsystem 110 of the surgical console 100, which brings the barcode area 289 into alignment with window 290 of LSBR 1002 in the surgical console 100. In step 404, the surgical console 100 attempts to decode the barcode 1016 while the visible light sources 1006a-d are on and the infrared light source 1030 remains off. If decoding the barcode 1016 is successful, the surgical console 100 proceeds with normal operating procedure in step 412. If decoding the barcode 1016 fails, for example when a spot 414 of external light is interfering with the barcode 1016 as seen in FIG. 15B, then in step 406, the visible light sources 1006a-d are turned off and the image sensor 1004 is activated in order to perform a test for detecting any external light which may be inadvertently shinning on the surgical cassette 200.

According to certain embodiments, the test or check for external light interference that is performed within step 406 is any one of the procedures 304A-F shown in FIGS. 9-14 and described above. According to certain other embodiments, step 406 comprises more than one of the procedures 304A-F shown in FIGS. 9-14 performed in any order with respect to one another. If external light interference is detected in step 406 by at least one of the procedures 304A-F shown in FIGS. 9-14, an ambient light advisory is generated for the user in step 408 to direct the user to remove the external light source and/or turn the surgical console 100 away from the external light source. After the user addresses the external light in step 411 and clears the advisory in step 415, the procedure returns to step 404, decode barcode. However if no external light interference is detected in step 406 by at least one of the procedures chosen from 304A-F shown in FIGS. 9-14, a barcode failure advisory is generated for the user in step 410. After user replaces cassette in step 413, the procedure returns to step 402 where a new cassette is coupled to the fluidics subsystem 110 of the surgical console 100.

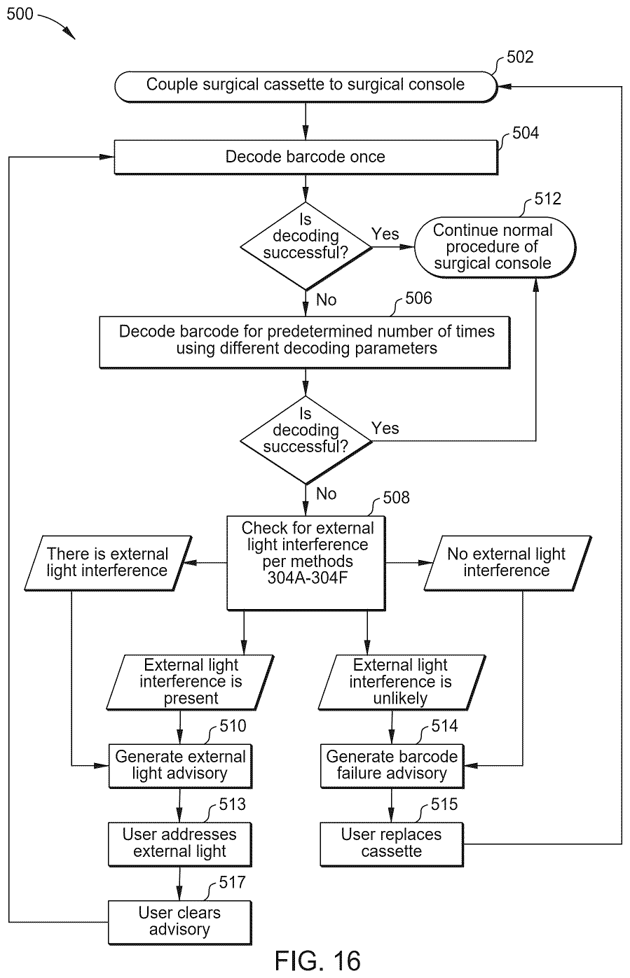

According to certain embodiments, the method of checking external light interference of the current invention is incorporated into a procedure 500 for coupling and connecting the surgical cassette 200 to the fluidics subsystem 110 of the surgical console 100, as seen in the flow chart of FIG. 16. The procedure begins with step 502 where a user couples the surgical cassette 200 to the fluidics subsystem 110 of the surgical console 100, which brings the barcode area 289 into alignment with window 290 of LSBR 1002 in the surgical console 100. In step 504, the surgical console 100 attempts to decode the barcode 1016 while the visible light sources 1006a-d are on and the infrared light source 1030 remains off. If decoding the barcode 1016 is successful, the surgical console 100 proceeds with normal operating procedure in step 512. If decoding the barcode 1016 initially fails (i.e., the initial attempt is unsuccessful), then in step 506 the surgical console 100 attempts to decode the barcode 1016 a predetermined number of times or instances. According to certain embodiments, step 506 comprises the surgical console 100 attempting to decode the barcode 1016 at least six times. According to certain embodiments, the exposure and/or gain of the image sensor 1004, the power or intensity of the visible light sources 1006a-d, and/or the decoding effort level of the algorithm for decoding the barcode 1016 are varied or adjusted between each subsequent attempt. If the barcode 1016 is successfully decoded in step 506, the surgical console 100 proceeds with normal operating procedure in step 512. However, if the barcode 1016 cannot be decoded after the predetermined number of attempts, the visible light sources 1006a-d are turned off and the image sensor 1004 is activated in order to perform a test for detecting any external light which may be inadvertently shinning on the surgical cassette 200 in step 508.

According to certain embodiments, the test or check for external light interference that is performed within step 508 is any one of the procedures 304A-F shown in FIGS. 9-14 and described above. According to certain other embodiments, step 508 comprises more than one of the procedures 304A-F shown in FIGS. 9-14 performed in any order with respect to one another. If external light interference is detected in step 508 by at least one of the procedures 304A-F shown in FIGS. 9-14, an external light advisory is generated for the user in step 510 to direct the user to remove the external light source and/or turn the surgical console 100 away from the external light source. After the user addresses the external light in step 513 and clears the advisory in step 517, the procedure returns to step 504, decode barcode. However, if no external light interference is detected or determined to be unlikely in step 508 by at least one of the procedures chosen from 304A-F shown in FIGS. 9-14, a barcode failure advisory is generated for the user in step 514. After user replaces cassette in step 515, the procedure returns to step 502 where a new cassette is coupled to the fluidics subsystem 110 of the surgical console 100.

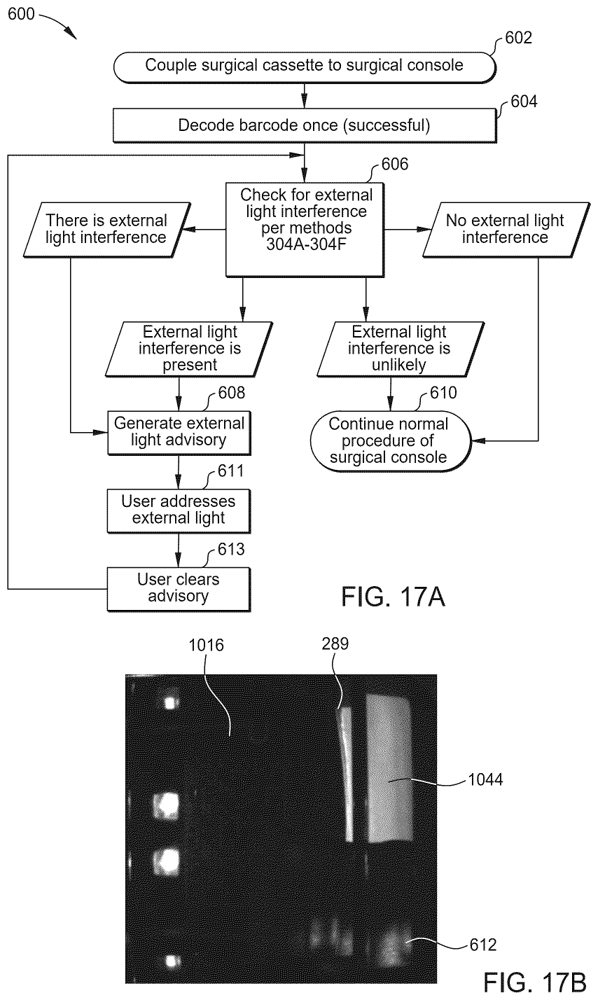

According to certain embodiments, the method of checking external light interference of the current invention is incorporated into a procedure 600 for coupling and connecting the surgical cassette 200 to the fluidics subsystem 110 of the surgical console 100, as seen in the flow chart of FIG. 17A. The procedure begins with step 602 where a user couples the surgical cassette 200 to the fluidics subsystem 110 of the surgical console 100, which brings the barcode area 289 into alignment with window 290 of LSBR 1002 in the surgical console 100. In step 604, the surgical console 100 decodes the barcode 1016 successfully while the visible light sources 1006a-d are on and the infrared light source 1030 remains off. Subsequently in step 606, the visible light sources 1006a-d are turned off and the image sensor 1004 is activated in order to perform a test for detecting any external light which may be inadvertently shinning on the surgical cassette 200.

According to certain embodiments, the test or check for external light interference that is performed within step 606 is any one of the procedures 304A-F shown in FIGS. 9-14 and described above. According to certain other embodiments, step 606 comprises more than one of the procedures 304A-F shown in FIGS. 9-14 performed in any order with respect to one another. If external light interference is detected in step 606 by at least one of the procedures 304A-F shown in FIGS. 9-14, for example when a spot 612 of external light is interfering with the fluid level window 1044 as seen in FIG. 17B, an external light advisory is generated for the user in step 608 to direct the user to remove the external light source and/or turn the surgical console 100 away from the external light source. After the user addresses the external light in step 611 and clears the advisory in step 613, the procedure returns to step 606. However, if no external light interference is detected or determined to be unlikely in step 606 by at least one of the procedures chosen from 304A-F shown in FIGS. 9-14, the surgical console 100 proceeds with normal operating procedure in step 610. By performing the check for external light interference in step 606 after decoding the barcode 1016 successfully in step 604, this ensures that no external light is present anywhere within the barcode area 289 of the surgical cassette 200, including the fluid level window 1044 that is offset or in a separate region than the barcode 1016.

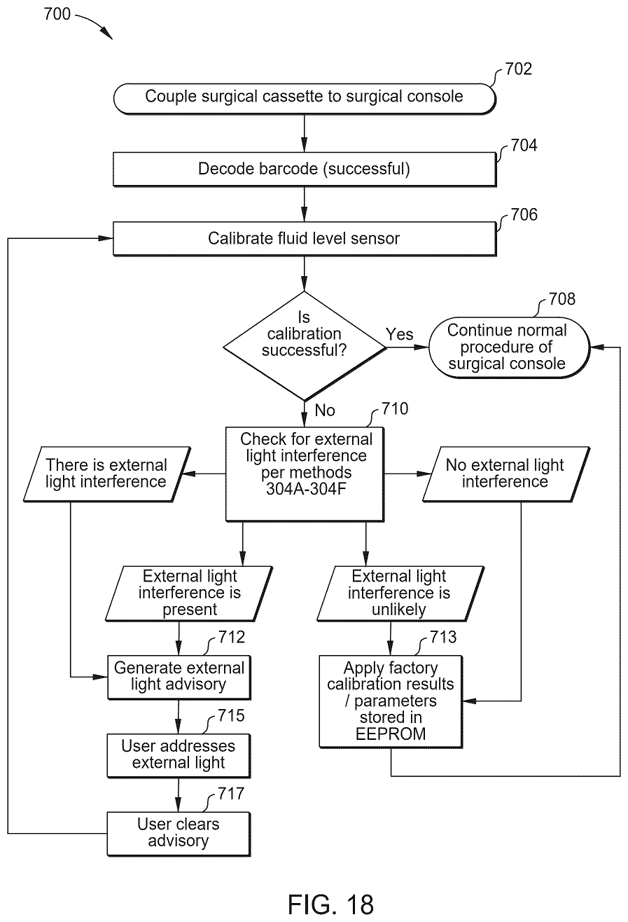

According to certain embodiments, the method of checking external light interference of the current invention is incorporated into a procedure 700 for coupling and connecting the surgical cassette 200 to the fluidics subsystem 110 of the surgical console 100, as seen in the flow chart of FIG. 18. The procedure begins with step 702 where a user couples the surgical cassette 200 to the fluidics subsystem 110 of the surgical console 100, which brings the barcode area 289 into alignment with window 290 of LSBR 1002 in the surgical console 100. In step 704, the surgical console 100 decodes the barcode 1016 successfully while the visible light sources 1006a-d are on and the infrared light source 1030 remains off. Subsequently in step 706, the surgical console 100 attempts to calibrate fluid level sensor functions of LSBR 1002 against the fluid level window 1044 within the barcode area 289. According to certain embodiments, such calibration is needed to determine location and size of the fluid level window 1044, and illumination intensity in gain value of the image sensor 1004 while exposure and LED power/current are fixed. If the calibration is successful, the surgical console proceeds with normal operating procedure in step 708. However, if the calibration in step 706 fails, the visible light sources 1006a-d and the infrared light source 1030 are turned off and the image sensor 1004 is activated in order to perform a test for detecting any external light which may be inadvertently shinning on the surgical cassette 200 in step 710.

According to certain embodiments, the test or check for external light interference that is performed within step 710 is any one of the procedures 304A-F shown in FIGS. 9-14 and described above. According to certain other embodiments, step 710 comprises more than one of the procedures 304A-F shown in FIGS. 9-14 performed in any order with respect to one another. If external light interference is detected in step 710 by at least one of the procedures 304A-F shown in FIGS. 9-14, an ambient light advisory is generated for the user in step 712 to direct the user to remove the external light source and/or turn the surgical console 100 away from the external light source. After the user addresses the external light in step 715 and clears the advisory in step 717, the procedure returns to step 706, calibrate fluid level sensor. However, if no external light interference is detected or determined to be unlikely in step 710 by at least one of the procedures chosen from 304A-F shown in FIGS. 9-14, the computer 103 of surgical console 100 applies factory calibration results/parameters stored in a memory of LSBR 1002 in step 713 and then proceeds with normal operating procedure in step 708. If external light shows up in or near the fluid level window 1044, calibration may fail. It is possible that factors other than external light may cause calibration failures, for example the surgical cassette 200 may already be filled with fluid when it is being reused. In this case, the size and location of the fluid level window 1044 cannot be determined, therefore requiring use of less accurate factory calibration results and parameters that are stored in the memory of LSBR 1002.

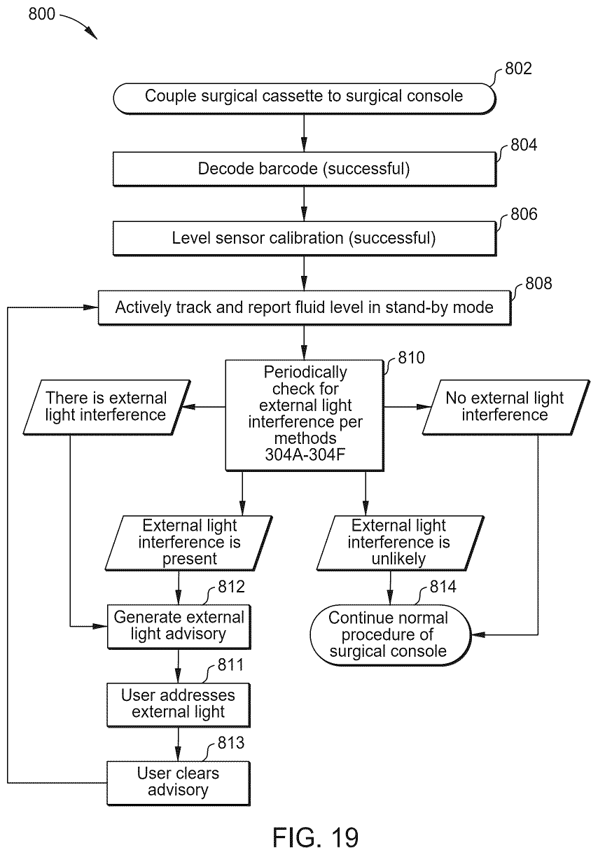

According to certain embodiments, the method of checking external light interference of the current invention is incorporated into a procedure 800 for coupling and connecting the surgical cassette 200 to the fluidics subsystem 110 of the surgical console 100, as seen in the flow chart of FIG. 19. The procedure begins with step 802 where a user couples the surgical cassette 200 to the fluidics subsystem 110 of the surgical console 100, which brings the barcode area 289 into alignment with window 290 of LSBR 1002 in the surgical console 100. In step 804, the surgical console 100 decodes the barcode 1016 successfully. Subsequently in step 806, the surgical console 100 successfully calibrates the fluid level sensor functions of LSBR 1002 against the fluid level window 1044. According to certain embodiments, after decoding the barcode 1016 and calibrating the fluid level sensor among other actions, basic set-up of the surgical console 100 is complete and the surgical console 100 then enters a stand-by mode in step 808 wherein the visible light sources 1006a-d are turned off and the infrared light source 1030 is turned on so as to continuously track and report the current fluid level within the fluid level window 1044 in real time. In step 810, while still in stand-by mode, the infrared light source 1030 is periodically turned off and the image sensor 1004 is activated in order to perform a test for detecting any external light which may be inadvertently shinning on the surgical cassette 200. According to certain embodiments, “periodic” or “periodically” refers to preselected durations of time which are predetermined or otherwise performed according to a predetermined schedule or command issued by the surgical console 100. According to certain embodiments, “continuous” or “continuously” refers to constant or uninterrupted performance of a task.

According to certain embodiments, the test or check for external light interference that is performed within step 810 is any one of the procedures 304A-F shown in FIGS. 9-14 and described above. According to certain other embodiments, step 810 comprises more than one of the procedures 304A-F shown in FIGS. 9-14 performed in any order with respect to one another. If external light interference is detected in step 810 by at least one of the procedures 304A-F shown in FIGS. 9-14, an external light advisory is generated for the user in step 812 to direct the user to remove the external light source and/or turn the surgical console 100 away from the external light source. After the user addresses the external light in step 811 and clears the advisory in step 813, the procedure returns to step 808, actively track and report fluid level in stand-by mode. However, if no external light interference is detected or determined to be unlikely in step 810 by at least one of the procedures chosen from 304A-F shown in FIGS. 9-14, the surgical console 100 proceeds with normal operating procedure in step 814.

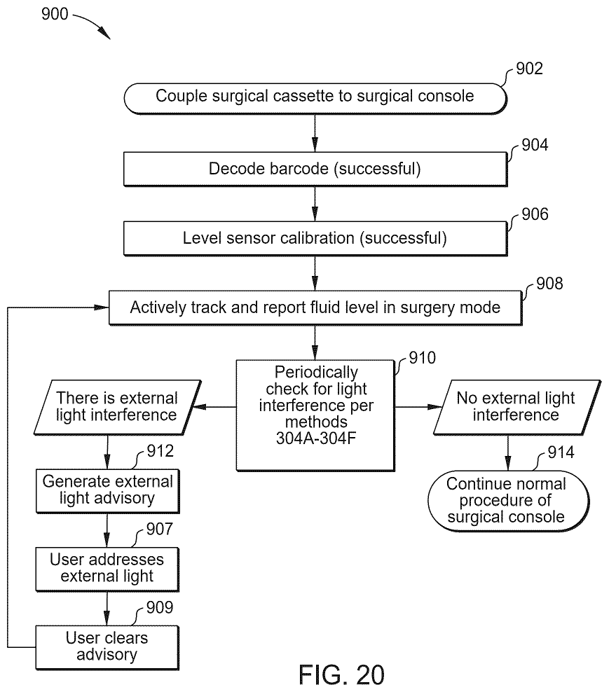

According to certain embodiments, the method of checking external light interference of the current invention is incorporated into a procedure 900 for coupling and connecting the surgical cassette 200 to the fluidics subsystem 110 of the surgical console 100, as seen in the flow chart of FIG. 20. The procedure begins with step 902 where a user couples the surgical cassette 200 to the fluidics subsystem 110 of the surgical console 100, which brings the barcode area 289 into alignment with window 290 of LSBR 1002 in the surgical console 100. After that, in step 904, the surgical console 100 decodes the barcode 1016 successfully. Subsequently in step 906, the surgical console 100 successfully calibrates the fluid level sensor functions of LSBR 1002 against fluid level window 1044. According to certain embodiments, after decoding the barcode 1016 and calibrating the fluid level senor among other actions, basic set-up of the surgical console 100 is complete and the surgical console 100 then enters a surgery mode in step 908 wherein the visible light sources 1006a-d are turned off and the infrared light source 1030 is turned on so as to continuously track and report the current fluid level within the fluid level window 1044 in real time during a surgery in step 908. In step 910, while still in surgery mode, the infrared light source 1030 is periodically turned off and the image sensor 1004 is activated in order to perform a test for detecting any external light which may be inadvertently shinning on the surgical cassette 200.

According to certain embodiments, the test or check for external light interference that is performed within step 910 is either one or both of the procedures 304A, and 304D shown in FIGS. 9 and 12, respectively, and described above. Because the surgical console 100 is in active surgery mode, only the procedure 304A as seen in FIG. 9 or the procedure 304D as seen in FIG. 12 are feasible given their use of analyzing single images. This is because analyzing multiple consecutive frames over a long duration to check for external light with the infrared light source 1030 off basically disables the fluid level sensor as the fluid level window 1044 cannot be seen for the same number of consecutive images or duration, thereby resulting in losing the ability to track real time fluid level during surgery. Conversely, single image checking external light interference is quick enough to be fit in between normal frames of the barcode area 289, or in certain other embodiments, can periodically replace a single normal frame of the barcode area 289, which is not disruptive to the continuous tracking of fluid level. According to certain embodiments, the image sensor 1004 is set to have an exposure of 0.5 ms, a gain of 3.0×, and a frame rate of 60 frames per second, which results 16.667 ms total time available for each frame/image. Therefore, as long as the total time for capturing and processing each frame/image including an exposure of 0.5 ms is less than half of 16.667 ms or 8 ms, an image for checking for of external light interference can be taken between two normal fluid level tracking images of barcode area 289.

According to certain embodiments, step 910 comprises the procedures 304A, 304D shown in FIGS. 9 and 12 performed in any order with respect to one another. If external light interference is detected in step 910 by at least one of the procedures 304A, 304D shown in FIG. 9 or 12, an ambient light advisory is generated for the user in step 912 to direct the user to remove the external light source and/or turn the surgical console 100 away from the external light source. After the user addresses the external light in step 907 and clears the advisory in step 909, the procedure returns to step 908, actively track and report fluid level in surgery mode. However, if no external light interference is detected in step 910 by at least one of the procedures 304A, 304D shown in FIG. 9 or 12, the surgical console 100 proceeds with normal operating procedure in step 914.

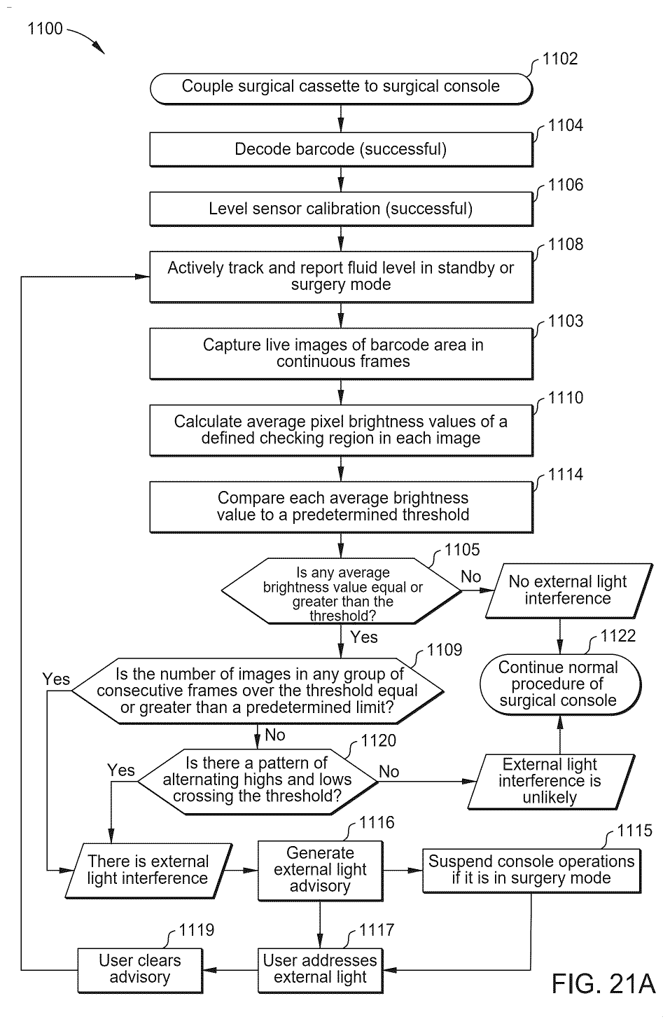

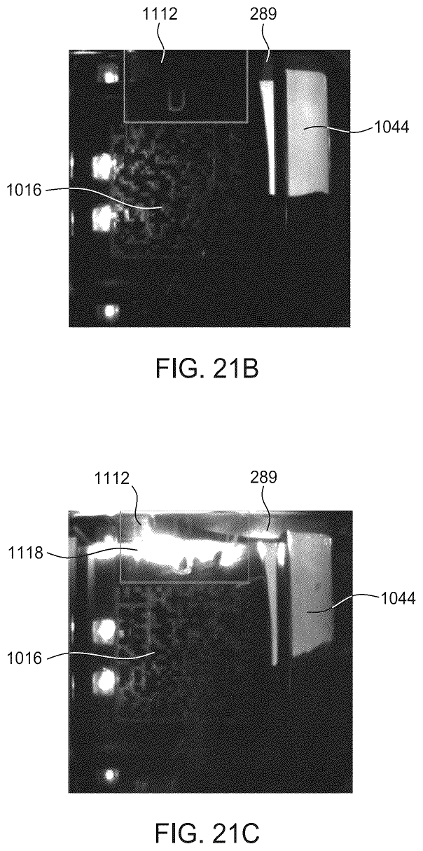

According to certain embodiments, the method of checking for external light interference of the current invention is incorporated into a procedure 1100 for coupling and connecting the surgical cassette 200 to the fluidics subsystem 110 of the surgical console 100, as seen in the flow chart of FIG. 21A. The procedure begins with step 1102 where a user couples the surgical cassette 200 to the fluidics subsystem 110 of the surgical console 100, which brings the barcode area 289 into alignment with window 290 of LSBR 1002 in the surgical console 100. After that, in step 1104, the surgical console 100 decodes the barcode 1016 successfully. Subsequently in step 1106, the surgical console 100 successfully calibrates the fluid level sensor functions of LSBR 1002 against fluid level window 1044.

According to certain embodiments, after decoding the barcode 1016 and calibrating the fluid level sensor among other actions, basic set-up of the surgical console 100 is complete and the surgical console 100 then enters either a stand-by mode or a surgery mode in step 1108 wherein the visible light sources 1006a-d are turned off and the infrared light source 1030 is turned on so as to continuously track and report the current fluid level within the fluid level window 1044 in real time. While a continuous series of live frames or images of the barcode area 289 are being captured and processed for fluid level tracking and reporting in step 1103, according to certain embodiments, a checking region 1112 defined within the barcode area 289 of each frame is being processed for checking for external light interference. The checking region 1112 is outside or away from both the barcode 1016 and the fluid level window 1044 and is typically dark during stand-by or surgical procedures, thereby increasing the likelihood of detecting external light therein. For example, according to certain embodiments, FIG. 21B shows the defined checking region 1112 above the barcode 1016 and to the left of the fluid level window 1044.

In step 1110, the computer 103 calculates an average pixel brightness value of the defined checking region 1112 for each frame or image. According to certain embodiments, steps 1108 and 1110 occur simultaneously so that active real-time tracking of the fluid level within the fluid level window 1044 is performed at the same time as checking for external light interference. In step 1114, the calculated average pixel brightness value of the defined checking region for each image is then compared against a predetermined threshold stored within the computer 103. According to certain embodiments, the predetermined threshold value is 10. In step 1105, if no average pixel brightness value of checking region of any frame is equal to or greater than the predetermined threshold, there is no external light interference, and the surgical console 100 then proceeds with normal operating procedure in step 1122. Alternatively, in step 1105, if there are calculated average pixel brightness values of the checking regions equal to or greater than the threshold value, then in step 1109 the computer 103 compares the frame number counts of every group of consecutive frames that have such average pixel brightness values that are equal to or greater than the predetermined threshold against a predetermined number limit, for example 16. If any of such frame number counts is equal to or greater than the predetermined number limit, then external light interference has been detected and an advisory image or message is sent to the user in step 1116 while the surgical console 100 automatically suspends fluid level dependent console operations in step 1115 if it is in surgery mode. After the user addresses the external light in step 1117 and clears the advisory in step 1119, the procedure returns to step 1108. Alternatively, if none of such frame number counts is equal to or greater than the predetermined number limit, then in step 1120 the computer 103 determines whether or not an alternating pattern of average pixel brightness values of the checking regions having highs and lows crossing the threshold exists.

According to certain embodiments, if the computer 103 detects such alternating pattern in step 1120, then external light interference has been detected and an advisory image or message is sent to the user in step 1116 while the surgical console 100 automatically suspends fluid level dependent console operations in step 1115 if it is in surgery mode. After the user addresses the external light in step 1117 and clears the advisory in step 1119, the procedure returns to step 1108. Alternatively if none such alternating pattern is found in step 1120, then external light interference is unlikely, the surgical console 100 proceeds with normal operating procedure in step 1122, FIG. 21C shows when a large area of external light 1118 is present within the defined checking region 1112. The brightness and size of the external light 1118 makes the average pixel brightness value of the checking region 1112 rise above the predetermined limit. Therefore if the external light 1118 lasts a minimum of consecutive frames, for example 16, or the external light 1118 runs in a long lasting on and off pattern, this method of checking for external light interference of the current invention will detect and determine it to be external light interference.

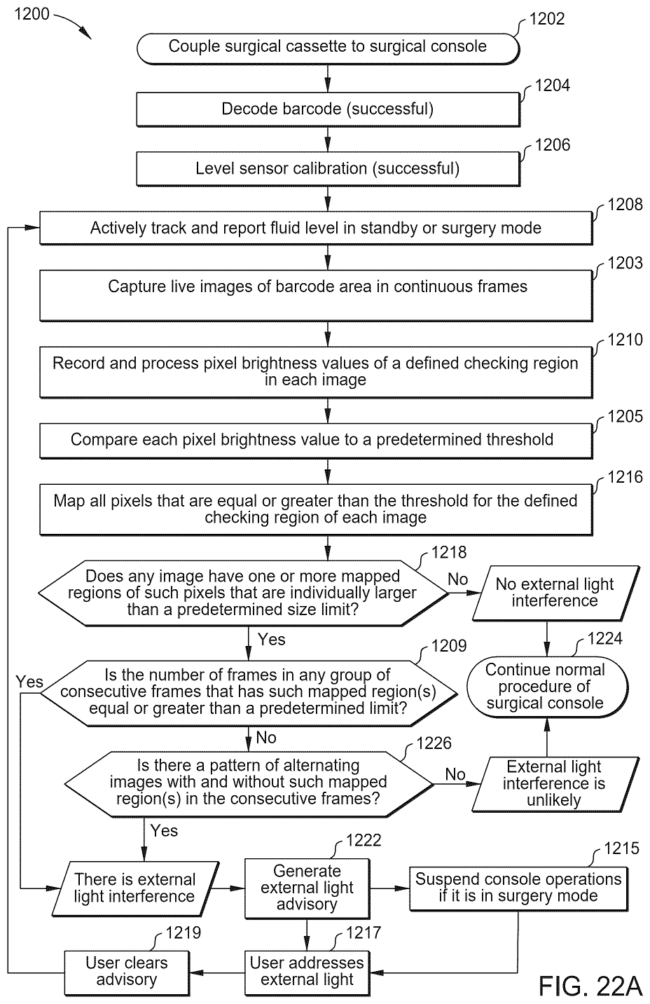

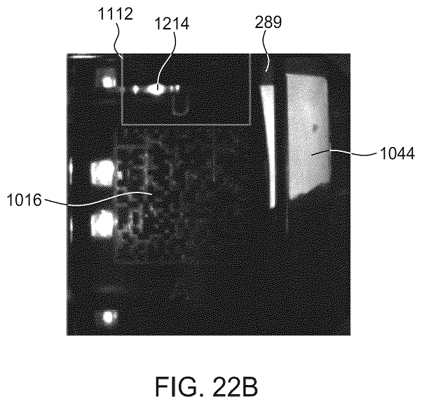

According to certain embodiments, the method of checking for external light interference of the current invention is incorporated into a procedure 1200 for coupling and connecting the surgical cassette 200 to the fluidics subsystem 110 of the surgical console 100, as seen in the flow chart of FIG. 22A. The procedure begins with step 1202 where a user couples the surgical cassette 200 to the fluidics subsystem 110 of the surgical console 100, which brings the barcode area 289 into alignment with window 290 of LSBR 1002 in the surgical console 100. In step 1204, the surgical console 100 decodes the barcode 1016 successfully. After that, in step 1206, the surgical console 100 successfully calibrates the fluid level sensor functions of LSBR 1002 against fluid level window 1044. According to certain embodiments, after decoding the barcode 1016 and calibrating the fluid level sensor among other actions, basic set-up of the surgical console 100 is complete and the surgical console 100 then enters either a stand-by mode or a surgery mode in step 1208 wherein the visible light sources 1006a-d are turned off and the infrared light source 1030 is turned on so as to continuously track and report the current fluid level within the fluid level window 1044 in real time.

While continuous live frames or images of the barcode area 289 are being captured and processed for fluid level tracking and reporting in step 1203, according to certain embodiments, a checking region 1112 is defined within the barcode area 289 of each frame that is being processed. The checking region 1112 is outside or away from both the barcode 1016 and the fluid level window 1044 and is typically dark during stand-by or surgical procedure, thereby increasing the likelihood of detecting external light therein. For example, according to certain embodiments, FIG. 22B shows the defined checking region 1112 above the barcode 1016 and to the left of the fluid level window 1044. In step 1210, the computer 103 records and processes brightness values for each of pixels contained within the defined checking region 1112 for each frame or image. According to certain embodiments, steps 1208 and 1210 occur simultaneously so that active real-time tracking of the fluid level within the fluid level window 1044 is performed at the same time as checking for external light interference. In step 1205, the brightness value for each pixel in the checking regions 1112 is then compared against a predetermined threshold stored within the computer 103.

Next, according to certain embodiments, a map of all pixels in the checking regions 1112, which have been determined to have a brightness value that is equal to or greater than the predetermined threshold is generated for each image in step 1216. The computer 103 then determines if the generated map corresponding to each image contains any regions therein that are individually larger than a predetermined size limit in step 1218. For example, as seen in FIG. 22B, an exemplary image of the barcode area 289 is shown wherein a local bright region 1214 of pixels that have been determined to have a pixel brightness value greater than the predetermined threshold is located inside the checking region 1112. The local bright region 1214 has a relatively large area or size that is over the predetermined size limit. According to certain embodiments, the predetermined size limit is a region size that is at least 5 pixels by 5 pixels. If no image has at least one such mapped region, there is no external light interference, the surgical console 100 proceeds with normal operating procedure in step 1224. If there are images having at least one such mapped region, in step 1209 the computer 103 compares the frame number counts of every group of consecutive frames that have such mapped regions against a predetermined number limit, for example 16. If any of such frame number counts is equal to or greater than the predetermined number limit, then external light interference has been detected and an advisory image or message is sent to the user in step 1222 while the surgical console 100 automatically suspends fluid level dependent console operations in step 1215 if it is in surgery mode. After the user addresses the external light in step 1217 and clears the advisory in step 1219, the procedure returns to step 1208. Alternatively, if none of such frame number counts is equal to or greater than the predetermined number limit, then in step 1226 the computer 103 determines whether or not a pattern of alternating images with and without such mapped regions exists.

According to certain embodiments, if the computer 103 detects such alternating pattern in step 1226, then external light interference has been detected and an advisory image or message is sent to the user in step 1222 while the surgical console 100 automatically suspends fluid level dependent console operations in step 1215 if it is in surgery mode. After the user addresses the external light in step 1217 and clears the advisory in step 1219, the procedure returns to step 1208. Alternatively if none such alternating pattern is found in step 1226, then external light interference is unlikely and the surgical console 100 proceeds with normal operating procedure in step 1224.

According to certain embodiments, in the methods of checking for external light interference of the current invention, the predetermined pixel brightness value threshold may be adjusted or changed according to the exposure and or gain settings of the image sensor 1004, and/or the amount of power or current running through the visible light sources 1006a-d and or the infrared light source 1030 if they are required to be turned on. Since pixel brightness values are directly affected by exposure, gain, and illumination, the higher the exposure, gain, or illumination, the higher the resulting pixel brightness value. According to certain embodiments, to operate the image sensor 1004 properly for decoding the barcode and for fluid level sensing, one of those three factors is adjustable while the other two are fixed. For example, according to certain embodiments where gain is an adjustable variable, the pixel brightness value threshold (T) is proportional to the gain value (G). In another embodiment, the pixel brightness value threshold (T) is determined according to a polynomial equation and or to a table that makes the threshold (T) increase when gain value (G) increases. An example of a polynomial equation and a table are given below.

$$T = 0 \text{.75} {G}^{⋀} 2 - 0 \text{.75} G + 55$$

TABLE 1

| Gain | 1 | 2 | 3 | 4 | 5 | 6 | 7 | 8 |
|---|---|---|---|---|---|---|---|---|
| Threshold | 55.0 | 56.5 | 59.5 | 64.0 | 70.0 | 77.5 | 86.5 | 97.0 |

Gain value is set during fluid level sensor calibration and based on the average pixel brightness value of the fluid level window 1044 when it is empty while exposure and the level of power/current in the infrared light sources 1030 remain fixed. Similarly, in other embodiments, the pixel brightness value threshold (T) may be proportional to exposure or illumination power instead of the gain value (G) and may be determined according to a polynomial equation and or to a table where exposure or illumination power is a variable.

Accordingly, improved ophthalmic surgical cassettes, means for being read by an image sensor disposed within the surgical console, and methods of use thereof are provided herein.

The foregoing description is provided to enable any person skilled in the art to practice the various embodiments described herein. Various modifications to these embodiments will be readily apparent to those skilled in the art, and the generic principles defined herein may be applied to other embodiments. Thus, the claims are not intended to be limited to the embodiments shown herein, but are to be accorded the full scope consistent with the language of the claims.

Certain features that are described in this specification in the context of separate implementations may also be implemented, in combination, in a single implementation. Conversely, various features that are described in the context of a single implementation may also be implemented in multiple implementations, separately, or in any suitable sub-combination. Moreover, although previously described features may be described as acting in certain combinations and even initially claimed as such, one or more features from a claimed combination may, in some cases, be excised from the combination, and the claimed combination may be directed to a sub-combination or variation of a sub-combination.

Particular implementations of the subject matter have been described. Other implementations, alterations, and permutations of the described implementations are within the scope of the following claims as will be apparent to those skilled in the art. While operations are depicted in the drawings or claims in a particular order, this should not be understood as requiring that such operations be performed in the particular order shown or in sequential order, or that all illustrated operations be performed (some operations may be considered optional) to achieve desirable results. In certain circumstances, multitasking or parallel processing (or a combination of multitasking and parallel processing) may be advantageous and performed as deemed appropriate.

Moreover, the separation or integration of various system modules and components in the previously described implementations should not be understood as requiring such separation or integration in all implementations. It should be understood that the described program components and systems may generally be integrated together in a single software product or packaged into multiple software products.

Accordingly, the previously described example implementations do not define or constrain the present disclosure. Other changes, substitutions, and alterations are also possible without departing from the spirit and scope of the present disclosure.

While the various steps in an embodiment method or process are presented and described sequentially, one of ordinary skill in the art will appreciate that some or all of the steps may be executed in different order, may be combined or omitted, and some or all of the steps may be executed in parallel. The steps may be performed actively or passively. The method or process may be repeated or expanded to support multiple components or multiple users within a field environment. Accordingly, the scope should not be considered limited to the specific arrangement of steps shown in a flowchart or diagram.

Unless defined otherwise, all technical and scientific terms used have the same meaning as commonly understood by one of ordinary skill in the art to which these systems, apparatuses, methods, processes and compositions belong.

In this disclosure, the terms “top”, “bottom”, “side”, “above”, “below”, “up”, “down”, “upward”, “downward”, “horizontal”, “vertical”, and the like do not refer to absolute directions. Instead, these terms refer to directions relative to a nonspecific plane of reference. This non-specific plane of reference may be vertical, horizontal, or other angular orientation.

The singular forms “a,” “an,” and “the” include plural referents, unless the context clearly dictates otherwise. Within a claim, reference to an element in the singular is not intended to mean “one and only one” unless specifically so stated, but rather “one or more.” Unless specifically stated otherwise, the term “some” refers to one or more.

Embodiments of the present disclosure may suitably “comprise”, “consist” or “consist essentially of” the limiting features disclosed, and may be practiced in the absence of a limiting feature not disclosed. As used here and in the appended claims, the words “comprise,” “has,” and “include” and all grammatical variations thereof are each intended to have an open, non-limiting meaning that does not exclude additional elements or steps.

“Optional” and “optionally” means that the subsequently described material, event, or circumstance may or may not be present or occur. The description includes instances where the material, event, or circumstance occurs and instances where it does not occur.

As used, the term “determining” encompasses a wide variety of actions. For example, “determining” may include calculating, computing, processing, deriving, investigating, looking up, for example, looking up in a table, a database or another data structure, and ascertaining. Also, “determining” may include receiving, for example, receiving information, and accessing, for example, accessing data in a memory. Also, “determining” may include resolving, selecting, choosing, and establishing.

When the word “approximately” or “about” are used, this term may mean that there may be a variance in value of up to +10%, of up to 5%, of up to 2%, of up to 1%, of up to 0.5%, of up to 0.1%, or up to 0.1%.

Ranges may be expressed as from about one particular value to about another particular value, inclusive. When such a range is expressed, it is to be understood that another embodiment is from the one particular value to the other particular value, along with all particular values and combinations thereof within the range.

As used, terms such as “first” and “second” are arbitrarily assigned and are merely intended to differentiate between two or more components of a system, an apparatus, or a composition. It is to be understood that the words “first” and “second” serve no other purpose and are not part of the name or description of the component, nor do they necessarily define a relative location or position of the component. Furthermore, it is to be understood that that the mere use of the term “first” and “second” does not require that there be any “third” component, although that possibility is envisioned under the scope of the various embodiments described.

As used, “a CPU,” “a processor,” “at least one processor” or “one or more processors” generally refers to a single processor configured to perform one or multiple operations or multiple processors configured to collectively perform one or more operations. In the case of multiple processors, performance the one or more operations could be divided amongst different processors, though one processor may perform multiple operations, and multiple processors could collectively perform a single operation. Similarly, “a memory,” “at least one memory” or “one or more memories” generally refers to a single memory configured to store data and/or instructions, multiple memories configured to collectively store data and/or instructions.

Although only a few example embodiments have been described in detail, those skilled in the art will readily appreciate that many modifications are possible in the example embodiments without materially departing from the disclosed scope as described. Accordingly, all such modifications are intended to be included within the scope of this disclosure as defined in the following claims.

What is claimed is:

1. A method for detecting external light interference within a surgical cassette, the method comprising:
checking for external light interference within a barcode area of the surgical cassette with an image sensor of a surgical console after the barcode area has been aligned with the image sensor,
if the external light interference is not detected or not likely to be present within the barcode area, attempting to decode a barcode within the barcode area, or
if the external light interference is detected within the barcode area, generating a first advisory notice for a user.

2. The method of claim 1, wherein attempting to decode the barcode within the barcode area comprises:
if the barcode is successfully decoded, permitting input from the user to be received for operating the surgical console; or
if the barcode is not successfully decoded, generating a second advisory notice for the user.

3. The method of claim 1, further comprising:
receiving input from the user to clear the first advisory notice; and
rechecking for the external light interference within the barcode area with the image sensor.

4. The method of claim 1, wherein checking for the external light interference within the barcode area with the image sensor comprises:
capturing an image of the barcode area;
calculating an average pixel brightness value for the image;
comparing the calculated average pixel brightness value for the image to a predetermined threshold; and
if the calculated average pixel brightness value for the image is lower than the predetermined threshold, detecting no external light interference or that the external light interference is not likely to be present within the barcode area, or
if the calculated average pixel brightness value for the image is equal to or higher than the predetermined threshold, detecting the external light interference within the barcode area.

5. The method of claim 4, further comprising:
capturing a plurality of images of the barcode area;
calculating an average pixel brightness value for each of the plurality of images;
comparing the calculated average pixel brightness value for each of the plurality of images to the predetermined threshold; and
if the calculated average pixel brightness value for each of the plurality of images is lower than the predetermined threshold, detecting no external light interference or that the external light interference is not likely to be present within the barcode area, or
if the calculated average pixel brightness value for each of the plurality of images is equal to or higher than the predetermined threshold, detecting the external light interference within the barcode area.

6. The method of claim 5, further comprising:
if the calculated average pixel brightness for at least one of the plurality of images is equal to or higher than the predetermined threshold, determining if there is a pattern of the calculated average pixel brightness value for the plurality of images repeatedly crossing the predetermined threshold,
if there is a pattern of the calculated average pixel brightness value for the plurality of images repeatedly crossing the predetermined threshold, detecting the external light interference within the barcode area, or
if there is no pattern of the calculated average pixel brightness value for the plurality of images repeatedly crossing the predetermined threshold:
recording a number of images in consecutive frames within the plurality of images having average pixel brightness values equal to or higher than the predetermined threshold;
comparing the number of images in consecutive frames within the plurality of images having average pixel brightness values equal to or higher than the predetermined threshold to a predetermined number limit; and
if the number of images in consecutive frames having average pixel brightness values equal to or higher than the predetermined threshold is equal to or higher than the predetermined number limit, detecting the external light interference within the barcode area, or
if the number of images in consecutive frames having average pixel brightness values equal to or higher than the predetermined threshold is lower than the predetermined number limit, detecting no external light interference or that the external light interference is not likely to be present within the barcode area.

7. The method of claim 1, wherein checking for the external light interference within the barcode area with the image sensor comprises:
capturing an image of the barcode area;
recording a brightness value for each pixel within the image;
comparing the brightness value of each pixel within the image to a predetermined threshold;
generating a map comprising pixels that have brightness values that are equal to or above the predetermined threshold; and
if the generated map does not comprise any mapped regions of pixels having brightness values equal to or above the predetermined threshold, which is larger than a predetermined size limit, detecting no external light interference or that the external light interference is not likely to be present within the barcode area, or
if the generated map comprises at least one mapped region of pixels having brightness values equal to or above the predetermined threshold, which is larger than the predetermined size limit, detecting the external light interference within the barcode area.

8. The method of claim 7, further comprising:
capturing a plurality of images of the barcode area;
recording a brightness value for each pixel within each of the plurality of images;
comparing the brightness value of each pixel within each of the plurality of images to a predetermined threshold;
generating a map comprising pixels that have brightness values that are equal to or above the predetermined threshold for each of the plurality of images; and
if no generated map comprises at least one mapped region of pixels having brightness values equal to or above the predetermined threshold, which is larger than the predetermined size limit, detecting no external light interference or that the external light interference is not likely to be present within the barcode area, or
if every generated map comprises the at least one mapped region of pixels having brightness values equal to or above the predetermined threshold, which is larger than the predetermined size limit, detecting the external light interference within the barcode area.

9. The method of claim 8, further comprising:
if at least one generated map comprises the at least one mapped region of pixels having brightness values equal to or above the predetermined threshold, which is larger than the predetermined size limit, determining if there is an alternating pattern of maps comprising the at least one mapped region of pixels having brightness values equal to or above the predetermined threshold which is larger than the predetermined size limit,
if there is a pattern, detecting the external light interference within the barcode area, or
if there is no pattern:
recording a frame number count for each group of consecutive maps comprising the at least one mapped region of pixels having brightness values equal to or above the predetermined threshold which is larger than the predetermined size limit;
comparing the frame number count of each group to a predetermined number limit; and
if any frame number count is larger than the predetermined number limit, detecting the external light interference within the barcode area, or
if no frame number count is larger than the predetermined number limit, detecting no external light interference or that the external light interference is not likely to be present within the barcode area.

10. The method of claim 1, further comprising performing an initial attempt to decode the barcode within the barcode area before performing the checking for the external light interference within the barcode area with the image sensor.

11. The method of claim 10, further comprising repeatedly attempting to decode the barcode within the barcode area a plurality of instances before checking for the external light interference if the initial attempt is unsuccessful,
if the barcode is successfully decoded during at least one of the plurality of instances, permitting input from the user to be received for operating the surgical console, or
if the barcode is not successfully decoded during any of the plurality of instances, performing the checking for external light interference within the barcode area with the image sensor.

12. The method of claim 1, further comprising:
attempting to decode the barcode prior to performing the checking for the external light interference within the barcode area with the image sensor;
after decoding the barcode and before performing the checking for the external light interference within the barcode area with the image sensor, attempting to calibrate a fluid level sensor against a fluid level window within the barcode area; and
if the attempt to calibrate the fluid level sensor is successful, detecting no external light interference or that the external light interference is not likely to be present within the barcode area, or
if the attempt to calibrate the fluid level sensor is unsuccessful, checking for the external light interference within the barcode area with the image sensor.

13. A method for checking for external light interference within a surgical cassette comprising:
decoding a barcode within a barcode area of the surgical cassette after aligning the barcode area with an image sensor of a surgical console;
calibrating a fluid level sensor of the surgical console against a fluid level window within the barcode area;
monitoring a fluid level within the fluid level window while the surgical console is in either a stand-by or a surgery mode;
periodically checking for external light interference within the barcode area with the image sensor while the surgical console is in either the stand-by or the surgery mode; and
if the external light interference is not detected within the barcode area, engaging in a surgical procedure by the surgical console, or
if the external light interference is detected within the barcode area, generating an advisory notice for a user.

14. The method of claim 13, further comprising:
continuously checking for the external light interference within the barcode area with the image sensor when the surgical console is in either the stand-by or the surgery mode, wherein continuously checking comprises:
capturing a plurality of consecutive live images of the barcode area, each of the plurality of live images comprising a defined checking region therein;
calculating an average pixel brightness value for the defined checking region within each of the plurality of live images;
comparing the calculated average pixel brightness value for the defined checking region within each of the plurality of live images to a predetermined threshold; and
if the calculated average pixel brightness value for the defined checking region within each of the plurality of live images is lower than the predetermined threshold, detecting no external light interference or that the external light interference is not likely to be present within the barcode area,
if the calculated average pixel brightness value for the defined checking region within at least one of the plurality of live images is equal to or higher than the predetermined threshold, counting a number of consecutive images having a calculated average pixel brightness value for the defined checking region within the plurality of live images that are equal to or higher than the predetermined threshold,
if the number of consecutive images is equal to or greater than a predetermined number limit, detecting the external light interference within the barcode area, or
if the number of consecutive frames is less than the predetermined number limit, determining if there is alternating pattern of the average pixel brightness value of the defined checking region repeatedly crossing the predetermined threshold within the plurality of live images,
if there is a pattern of the average pixel brightness value of the defined checking region repeatedly crossing the predetermined threshold within the plurality of live images, detecting the external light interference within the barcode area, or
if there is no pattern of the average pixel brightness value of the defined checking region repeatedly crossing the predetermined threshold within the plurality of live images, detecting no external light interference or that the external light interference is not likely to be present within the barcode area.

15. The method of claim 13, wherein periodically checking for the external light interference within the barcode area with the image sensor in either the stand-by or the surgery mode comprises:
capturing a plurality of consecutive live images of the barcode area, each of the plurality of live images comprising a defined checking region therein;
calculating a brightness value for each pixel within the defined checking region of each of the plurality of live images;
comparing the brightness value of each pixel within the defined checking region of each of the plurality of live images to a predetermined threshold;
generating a map comprising pixels within the defined checking region having brightness values equal to or above the predetermined threshold for each of the plurality of live images; and
if none of the plurality of live images comprises at least one mapped region of pixels within the defined checking region having brightness values equal to or above the predetermined threshold, which is individually larger than a predetermined size limit, detecting no external light interference or that the external light interference is not likely to be present within the barcode area, or
if at least one of the plurality of live images comprises the at least one mapped region of pixels within the defined checking region having pixel brightness values equal to or above the predetermined threshold, which is individually larger than the predetermined size limit, counting a number of consecutive images having the at least one mapped region of pixels within the defined checking region having brightness values equal to or above the predetermined threshold which is individually larger than the predetermined size limit,
if the number of consecutive images is equal to or greater than a predetermined number limit, detecting the external light interference within the barcode area, or
if the number of consecutive frames is less than the predetermined number limit, determining if there is alternating pattern of the at least one mapped region of pixels within the defined checking region having brightness values equal to or above the predetermined threshold, which is individually larger than the predetermined size limit, repeatedly crossing the predetermined threshold within the plurality of live images,
if there is a pattern of the at least one mapped region of pixels within the defined checking region having brightness values equal to or above the predetermined threshold, which is individually larger than the predetermined size limit, repeatedly crossing the predetermined threshold within the plurality of live images, detecting the external light interference within the barcode area, or
if there is no pattern of the at least one mapped region of pixels within the defined checking region having brightness values equal to or above the predetermined threshold, which is individually larger than the predetermined size limit, repeatedly crossing the predetermined threshold within the plurality of live images, detecting no external light interference or that the external light interference is not likely to be present within the barcode area.
